(19) United States
(12) Patent Application Publication
(10) Pub. No.: US 2019/0169689 A1
(43) Pub. Date: Jun. 6, 2019

(54) COMPOSITIONS AND METHODS FOR ANALYZING NUCLEIC ACIDS ASSOCIATED WITH AN ANALYTE

(71) Applicant: CDI LABORATORIES, INC., Mayaguez, PR (US)

(72) Inventors: Heng ZHU, Mayaguez, PR (US); Ignacio PINO, Mayaguez, PR (US)

(21) Appl. No.: 16/324,836

(22) PCT Filed: Aug. 11, 2017
(86) PCT No.: PCT/US17/46519

§ 371 Date: Feb. 11, 2019

Related U.S. Application Data
(60) Provisional application No. 62/374,360, filed on Aug. 12, 2016.

(51) Int. Cl. C12Q 1/6876; C12Q 1/6834; C12Q 1/6869

(52) U.S. Cl. CPC C12Q 1/6876; C12Q 1/6834; C12Q 2600/166; C12Q 2600/16; C12Q 1/6869

(57) ABSTRACT

This disclosure provides compositions and methods for analyzing a nucleic acid associated with an analyte.

### CROSS-REFERENCE

This application claims priority to U.S. Provisional Application No. 62/374,360, filed Aug. 12, 2016; which is incorporated herein by reference in its entirety.

### BACKGROUND

Interactions between analytes and nucleic acids can have a significant impact on the translation of proteins encoded by the nucleic acid. In one example, certain combinations of post-translational modifications on histone tails serve as the mechanism to recruit other proteins, such as histone modification enzymes, which act to alter chromatin structure actively or to promote transcription. Accordingly, dysregulation of such mechanisms of transcriptional regulation can have negative consequences, resulting in and affecting the progression of many diseases such as cancer. Development of enabling technologies suitable for detecting or characterizing the effects of these interactions can allow for the prognostication of a given disease.

Traditional methods of analyzing the interactions between analytes and nucleic acids are limited. Chromatin immunoprecipitation sequencing (ChIP-seq) is one such technique that has been developed. However, in the example of post translational modification on histone tails as described above, ChIP-seq technology can be capable of surveying only a single post-translational modification at a time. Therefore, whether the outcome of transcription of a given gene is dependent on a particular combination of post-translational modifications could not be tested at the single nucleosome level. The present disclosure has several practical applications, providing compositions and methods for analyzing a nucleic acid associated with an analyte.

### BRIEF SUMMARY

This disclosure provides compositions and methods. In some aspects, this disclosure provides compositions comprising a first probe. In some embodiments, a first probe can comprise a first tag. In some embodiments a first tag can comprise a polynucleotide comprising a region for attaching to a first end of a nucleic acid. In some aspects, this disclosure provides compositions comprising a second probe. In some embodiments, a second probe can comprise a second tag. In some embodiments, a second tag can comprise a polynucleotide comprising a region for attaching to a second end of a nucleic acid. In some embodiments, a first probe can have an affinity to a first binding site on an analyte and a second probe can have an affinity to a second binding site on an analyte. In some embodiments, a first probe can have an affinity to a first binding site on an analyte. In some embodiments, a second probe can have an affinity to a second binding site on an analyte. In some embodiments, a first probe and the second probe can be in spatial proximity. In some embodiments, a first probe can be associated with a substrate. In some embodiments, a second probe can be associated with a substrate. In some embodiments, a first probe can be associated with a substrate and a second probe can be associated with a substrate. In some embodiments, a first probe can be associated with a substrate. In some embodiments, a second probe can be associated with a substrate. In some embodiments, a first tag can be double stranded. In some embodiments, a first tag can be double stranded where associated with a first probe. In some embodiments, a second tag is double stranded. In some embodiments, a second tag can be double stranded where associated with a second probe. In some embodiments, a first tag can be double stranded where associated with a first probe and a second tag can be double stranded where associated with a second probe. In some embodiments, a first probe can be associated with a substrate. In some embodiments, a first tag can be double stranded where associated with a first probe. In some embodiments, a first probe can be associated with a substrate, and a first tag can be double stranded where associated with a first probe. In some embodiments, a first probe can be associated with a substrate, and a second tag can be double stranded where associated with a second probe. In some embodiments, a second tag can be double stranded where associated with a second probe. In some embodiments, a first probe can be associated with a substrate, and a first tag can be double stranded where associated with a first probe and a second tag can be double stranded where associated with a second probe. In some embodiments, a second probe can be associated with a substrate. In some embodiments, a second probe can be associated with a substrate, and a first tag can be double stranded where associated with a first probe. In some embodiments, a second probe can be associated with a substrate, and a second tag can be double stranded where associated with a second probe. In some embodiments, a second probe can be associated with a substrate, and a first tag can be double stranded where associated with a first probe and a second tag can be double stranded where associated with a second probe. In some embodiments, a first probe can be associated with a substrate. In some embodiments, a first probe can be associated with a substrate and a second probe can be associated with a substrate, and a first tag can be double stranded where associated with a first probe. In some embodiments, a first probe can be associated with the substrate and the second probe can be associated with a substrate, and a second tag can be double stranded where associated with a second probe. In some embodiments, a first probe can be associated with a substrate and a second probe can be associated with a substrate, and a first tag can be double stranded where associated with a first probe and a second tag can be double stranded where associated with a second probe. In some embodiments, a first probe can be associated with a solid substrate. In some embodiments, a second probe can be associated with a solid substrate. In some embodiments, a solid substrate can be planar. In some embodiments, a substrate can be an array. In other embodiments, a solid substrate can be spherical. In some embodiments, a spherical solid substrate can be a bead. In some embodiments, at least a portion of a solid substrate can be coated. In some embodiments, at least a portion of a solid substrate can be contacted with at least one of a polymer or a first binding partner. In some embodiments, a polymer or a first binding partner can have an affinity for a second binding partner. In some embodiments, a polymer can be selected from the group of polyethylene glycol, polymethacrylate, polymethylmethacrylate, polyethylenimine, polyvinyl alcohol, polyvinyl acetate, polystyrene, polyglutaraldehyde, polyacrylamide, agarose, chitosan, alginate, or a combination thereof. In some embodiments comprising a first binding partner can be selected from a group of immunoglobulin-binding protein, calmodulin, glutathione, glutathione S-transferase (GST), streptavidin, avidin, maltose-binding protein, a His tag, or a combination thereof. In some embodiments, a second binding partner can be selected from the group of immunoglobulin-binding protein, calmodulin, glutathione, glutathione S-transferase (GST), streptavidin, avidin, maltose-binding protein, a His tag, or a combination thereof. In some embodiments, a immunoglobulin-binding protein can be Protein A or Protein G. In some embodiments, each of a first probe and a second probe can comprise at least one of a binding partner of the polymer or a second binding partner. In some embodiments a first binding partner can be GST and a first probe and a second probe can comprise glutathione. In some embodiments, a solid substrate can be magnetic. In some embodiments, a magnetic solid substrate can comprise magnetite, maghemitite, FePt, SrFe, iron, cobalt, nickel, chromium dioxide, ferrites, or a mixture thereof. In some embodiments, a solid substrate can be nonmagnetic. In some embodiments, a first probe can comprise a first antibody or a fragment thereof. In some embodiments, a first antibody or fragment thereof can comprise at least one of a binding partner of a polymer or a second binding partner. In some embodiments, a second probe can comprise a second antibody or a fragment thereof. In some embodiments, a second antibody or fragment thereof can comprise at least one of a binding partner of a polymer or a second binding partner. In some embodiments, a first antibody or a second antibody can be a monoclonal, recombinant, polyclonal, chimeric, humanized, bispecific antibody, or a fragment thereof. In some embodiments, a first antibody or a second antibody can be isolated or purified from a hybridoma. In some embodiments, a first probe can be conjugated with a first tag. In some embodiments, a second probe can be conjugated with a second tag. In some embodiments, a first antibody or a fragment thereof can be conjugated with a first tag. In some embodiments, a second antibody or the fragment thereof can be conjugated with a second tag. In some embodiments, a first antibody or a fragment thereof can be conjugated with a first tag and a second antibody or the fragment thereof can be conjugated with a second tag. In some embodiments, the first tag can be double stranded. In some embodiments, a second tag can be double stranded. In some embodiments, a second tag can be single stranded. In some embodiments, a first tag can comprise a first cleavage site. In some embodiments, a second tag can comprises a second cleavage site. In some embodiments, a first cleavage site and a second cleavage site can be endonuclease recognition sites. In some embodiments, the endonuclease site can comprise a type II endonuclease recognition site. In some embodiments, a type II endonuclease recognition site can be a BsaI recognition site. In some embodiments, a first tag can comprise a first barcode. In some embodiments, a second tag can comprise a second barcode. In some embodiments, a first barcode can comprise about 1 to about 50 nucleotides. In some embodiments, a second barcode can comprise about 1 to about 50 nucleotides. In some embodiments, a first tag can comprise a first primer binding site. In some embodiments, a second tag can comprise a second primer binding site. In some embodiments, a first tag can comprise a first primer binding site, and a second tag can comprise a second primer binding site. In some embodiments, a first probe can be uniquely identifiable by a first barcode. In some embodiments, a second probe can be uniquely identifiable by a second barcode. In some embodiments, a first polynucleotide and/or a second polynucleotide can be DNA. In some embodiments, a first polynucleotide and/or a second polynucleotide can be RNA. In some embodiments, a first polynucleotide and/or the second polynucleotide can be a hybrid of DNA and RNA. In some embodiments, an analyte can comprise a first biological molecule. In some embodiments, a first biological molecule can be a protein, a carbohydrate, a lipid, or a nucleic acid. In some embodiments, an analyte can comprise a first protein. In some embodiments, a first protein can comprise a first modified residue. In some embodiments, a first protein can comprise a first modified residue and a second modified residue. In some embodiments, a first probe can bind to an antigen comprising a first modified residue. In some embodiments, a second probe can bind to an antigen comprising a second modified residue. In some embodiments, a first probe can bind to an antigen comprising a first modified residue and a second probe can bind to an antigen comprising a second modified residue. In some embodiments, a modification on a first modified residue can be methylation, phosphorylation, acetylation, ubiquitylation, sumoylation, or a combination thereof. In some embodiments, a modification on a second modified residue can be methylation, phosphorylation, acetylation, ubiquitylation, sumoylation, or a combination thereof. In some embodiments, a first protein can be a histone. In some embodiments, a histone can be modified. In some embodiments, a histone modification can be methylation, acetylation, or a combination thereof. In some embodiments, a histone can be histone 3. In some embodiments, a histone can be modified at a residue. In some embodiments, a histone can be modified at a lysine residue. In some embodiments, an analyte can comprise a second protein. In some embodiments, a first protein and/or a second protein can comprise a transcription factor. In some embodiments, a first protein and/or a second protein can form a dimer. In some embodiments, a first protein can comprise a first binding site. In some embodiments, a second protein can comprise a second binding site. In some embodiments, a first protein can comprise a first binding site and a second protein can comprise a second binding site. In some embodiments, an analyte can be associated with a nucleic acid. In some embodiments, a nucleic acid comprises genomic DNA. In some embodiments, a nucleic acid can be intracellular or extracellular. In some embodiments, a nucleic acid can be RNA, DNA, or a hybrid thereof. In some embodiments, any of the compositions disclosed herein can be in the form of an array, performed in liquid phase or solid phase.

In some aspects, this disclosure provides methods comprising contacting a sample comprising a nucleic acid associated with an analyte with a first probe. In some embodiments, a first probe can comprise a first tag. In some embodiments, a first tag can comprise a polynucleotide. In some embodiments, a polynucleotide can comprise a region for attaching to a first end of a nucleic acid. In some embodiments, a second probe can comprise a second tag. In some embodiments, a second tag can comprise a polynucleotide. In some embodiments, a polynucleotide can comprise a region for attaching to a second end of a nucleic acid. In some embodiments, a second probe can comprise a second tag comprising a polynucleotide comprising a region for attaching to a second end of a nucleic acid. In some embodiments, a first probe can have an affinity to a first binding site on an analyte. In some embodiments, a second probe can have an affinity to a second binding site on an analyte. In some embodiments, a first probe can have an affinity to a first binding site on an analyte and a second probe can have an affinity to a second binding site on an analyte. In some embodiments, a first probe and a second probe can be in spatial proximity. In some embodiments, a first probe can be associated with a substrate. In some embodiments, a second probe can be associated with a substrate. In some embodiments, a first probe can be associated with a substrate and a second probe can be associated with the same or different substrate. In some embodiments, a first tag can be double stranded. In some embodiments, a first tag can be double stranded where associated with a first probe. In some embodiments, a second tag can be double stranded. In some embodiments, a second tag can be double stranded where associated with a second probe. In some embodiments, a first tag can be double stranded where associated with a first probe and a second tag can be double stranded where associated with a second probe. In some embodiments, a first probe can be associated with a substrate, and a first tag can be double stranded where associated with a first probe. In some embodiments, a first probe can be associated with a substrate, and a second tag can be double stranded where associated with a second probe. In some embodiments, a first probe can be associated with a substrate, and a first tag can be double stranded where associated with a first probe and a second tag can be double stranded where associated with a second probe. In some embodiments, a second probe can be associated with a substrate, and a first tag can be double stranded where associated with a first probe. In some embodiments, a second probe can be associated with a substrate, and a second tag can be double stranded where associated with a second probe. In some embodiments, a second probe can be associated with a substrate, and a first tag can be double stranded where associated with a first probe and a second tag can be double stranded where associated with a second probe. In some embodiments, a first probe can be associated with a substrate and a second probe can be associated with a substrate, and a first tag can be double stranded where associated with a first probe. In some embodiments, a first probe can be associated with a substrate and a second probe can be associated with a substrate, and a second tag can be double stranded where associated with a second probe. In some embodiments, a first probe can be associated with a substrate and a second probe can be associated with a substrate, and a first tag can be double stranded where associated with a first probe and a second tag can be double stranded where associated with a second probe. In some embodiments, a sample can be a biological sample. In some embodiments, a biological sample can be selected from amniotic fluid, blood plasma, blood serum, breast milk, cells, cancer cells, tumor cells, cerebrospinal fluid, saliva, semen, synovial fluid, tears, tissue, cancer tissue, tumor tissue, urine, white blood cells, whole blood, and any fraction thereof. In some embodiments, a nucleic acid can be an intracellular nucleic acid. In some embodiments, a nucleic acid can be an extracellular nucleic acid. In some embodiments, a nucleic acid can be DNA. In some embodiments, a nucleic acid can be RNA. In some embodiments, a nucleic acid can be a hybrid of DNA and RNA. In some aspects, the methods disclosed herein can further comprise cross-linking a nucleic acid to an analyte. In some aspects, the methods disclosed herein can further comprise cross-linking a nucleic acid to an analyte using a cross-linking agent. In some aspects, the methods disclosed herein can comprise modifying a nucleic acid. In some embodiments, modifying a nucleic acid can comprise generating a single stranded overhang at the first end of a nucleic acid or at a second end of a nucleic acid. In some aspects, the methods disclosed herein can comprise extracting a nucleic acid associated with an analyte from a sample. In some embodiments, a nucleic acid associated with an analyte can be extracted from a sample by contacting the sample with an extraction complex. In some embodiments, an extraction complex can comprise an extraction moiety. In some embodiments, an extraction complex can comprise an oligonucleotide. In some embodiments, an extraction complex can comprise an extraction moiety and an oligonucleotide. In some embodiments, an extraction complex can comprise an extraction moiety and an oligonucleotide, wherein the extraction complex binds to a nucleic acid. In some embodiments, at least one of a first probe binds to a first binding site on an analyte or a second probe binds to a second binding site on an analyte. In some aspects, the methods disclosed herein can comprise attaching a first tag to a first end of a nucleic acid associated with an analyte. In some embodiments, the method can comprise attaching a second tag to a second end of a nucleic acid associated with an analyte. In some embodiments, the method can comprise attaching a first tag to a first end of a nucleic acid associated with an analyte and attaching a second tag to a second end of a nucleic acid associated with an analyte. In some aspects, the methods disclosed herein can comprise analyzing a nucleic acid. In some embodiments, analyzing a nucleic acid can comprise at least one of amplifying a nucleic acid or sequencing a nucleic acid. In some embodiments, sequencing can comprise multiplex sequencing. In some embodiments, amplifying can comprise polymerase chain reaction. In some embodiments of the methods disclosed herein, a substrate can be an array. In some embodiments, a substrate can be a solid substrate. In some embodiments, a solid substrate can be planar. In other embodiments, a solid substrate can be spherical. In some embodiments, a spherical solid substrate can be a bead. In some embodiments, at least a portion of a solid substrate can be coated. In some embodiments, at least a portion of a solid substrate can be contacted with at least one of a polymer or a first binding partner. In some embodiments, a polymer or a first binding partner can have an affinity for a second binding partner. In some embodiments, a polymer can be selected from the group of polyethylene glycol, polymethacrylate, polymethylmethacrylate, polyethylenimine, polyvinyl alcohol, polyvinyl acetate, polystyrene, polyglutaraldehyde, polyacrylamide, agarose, chitosan, alginate, or a combination thereof. In some embodiments comprising a first binding partner can be selected from a group of immunoglobulin-binding protein, calmodulin, glutathione, glutathione S-transferase (GST), streptavidin, avidin, maltose-binding protein, a His tag, or a combination thereof. In some embodiments, a second binding partner can be selected from the group of immunoglobulin-binding protein, calmodulin, glutathione, glutathione S-transferase (GST), streptavidin, avidin, maltose-binding protein, a His tag, or a combination thereof. In some embodiments, a immunoglobulin-binding protein can be Protein A or Protein G. In some embodiments, each of a first probe and a second probe can comprise at least one of a binding partner of the polymer or a second binding partner. In some embodiments a first binding partner can be GST and a first probe and a second probe can comprise glutathione. In some embodiments, a substrate can be magnetic. In some embodiments, a magnetic solid substrate can comprise magnetite, maghemitite, FePt, SrFe, iron, cobalt, nickel, chromium dioxide, ferrites, or a mixture thereof. In some embodiments, a solid substrate can be nonmagnetic. In some embodiments, a first probe can comprise a first antibody or a fragment thereof. In some embodiments, a first antibody or fragment thereof can comprise at least one of a binding partner of a polymer or a second binding partner. In some embodiments, a second probe can comprise a second antibody or a fragment thereof. In some embodiments, a second antibody or fragment thereof can comprise at least one of a binding partner of a polymer or a second binding partner. In some embodiments, a first antibody or a second antibody can be a monoclonal, recombinant, polyclonal, chimeric, humanized, bispecific antibody, or a fragment thereof. In some embodiments, a first antibody or a second antibody can be isolated or purified from a hybridoma. In some embodiments, a first probe can be conjugated with a first tag. In some embodiments, a second probe can be conjugated with a second tag. In some embodiments, a first antibody or a fragment thereof can be conjugated with a first tag. In some embodiments, a second antibody or the fragment thereof can be conjugated with a second tag. In some embodiments, a first antibody or a fragment thereof can be conjugated with a first tag and a second antibody or the fragment thereof can be conjugated with a second tag. In some embodiments, the first tag can be double stranded. In some embodiments, a second tag can be double stranded. In some embodiments, a second tag can be single stranded. In some embodiments, a first tag can comprise a first cleavage site. In some embodiments, a second tag can comprises a second cleavage site. In some embodiments, a first cleavage site and a second cleavage site can be endonuclease recognition sites. In some embodiments, the endonuclease site can comprise a type II endonuclease recognition site. In some embodiments, a type II endonuclease recognition site can be a BsaI recognition site. In some embodiments, a first tag can comprise a first barcode. In some embodiments, a second tag can comprise a second barcode. In some embodiments, a first barcode can comprise about 1 to about 50 nucleotides. In some embodiments, a second barcode can comprise about 1 to about 50 nucleotides. In some embodiments, a first tag can comprise a first primer binding site. In some embodiments, a second tag can comprise a second primer binding site. In some embodiments, a first tag can comprise a first primer binding site, and a second tag can comprise a second primer binding site. In some embodiments, a first probe can be uniquely identifiable by a first barcode. In some embodiments, a second probe can be uniquely identifiable by a second barcode. In some embodiments, a first polynucleotide and/or a second polynucleotide can be DNA. In some embodiments, a first polynucleotide and/or a second polynucleotide can be RNA. In some embodiments, a first polynucleotide and/or the second polynucleotide can be a hybrid of DNA and RNA. In some embodiments, an analyte can comprise a first biological molecule. In some embodiments, a first biological molecule can be a protein, a carbohydrate, a lipid, or a nucleic acid. In some embodiments, an analyte can comprise a first protein. In some embodiments, a first protein can comprise a first modified residue. In some embodiments, a first protein can comprise a first modified residue and a second modified residue. In some embodiments, a first probe can bind to an antigen comprising a first modified residue. In some embodiments, a second probe can bind to an antigen comprising a second modified residue. In some embodiments, a first probe can bind to an antigen comprising a first modified residue and a second probe can bind to an antigen comprising a second modified residue. In some embodiments, a modification on a first modified residue can be methylation, phosphorylation, acetylation, ubiquitylation, sumoylation, or a combination thereof. In some embodiments, a modification on a second modified residue can be methylation, phosphorylation, acetylation, ubiquitylation, sumoylation, or a combination thereof. In some embodiments, a first protein can be a histone. In some embodiments, a histone can be modified. In some embodiments, a histone modification can be methylation, acetylation, or a combination thereof. In some embodiments, a histone can be histone 3. In some embodiments, a histone can be modified at a residue. In some embodiments, a histone can be modified at a lysine residue. In some embodiments, an analyte can comprise a second protein. In some embodiments, a first protein and/or a second protein can comprise a transcription factor. In some embodiments, a first protein and/or a second protein can form a dimer. In some embodiments, a first protein can comprise a first binding site. In some embodiments, a second protein can comprise a second binding site. In some embodiments, a first protein can comprise a first binding site and a second protein can comprise a second binding site. In some embodiments, an analyte can be associated with a nucleic acid. In some embodiments, a nucleic acid comprises genomic DNA. In some embodiments, a nucleic acid can be intracellular or extracellular. In some embodiments, a nucleic acid can be RNA, DNA, or a hybrid thereof. In some embodiments, any of the methods disclosed herein can be performed in liquid phase or solid phase. In some embodiments, any of the methods disclosed herein can be performed as a liquid phase assay or as a solid phase assay.

In some aspects, this disclosure provides methods comprising extracting an analyte from a sample by contacting the sample with an extraction complex comprising an extraction moiety and an oligonucleotide. In some embodiments, an extraction complex can bind to a nucleic acid. In some aspects, this disclosure provides methods comprising contacting an extracted analyte with a first probe that has an affinity to a first binding site on the analyte, and a second probe that has an affinity to a second binding site on the analyte. In some embodiments, a first probe can comprise a first tag comprising a first polynucleotide comprising a region for attaching to a first end of the nucleic acid, and a second probe can comprise a second tag comprising a second polynucleotide comprising a region for attaching to a second end of the nucleic acid. In some embodiments, a first probe and a second probe can be in spatial proximity. In some aspects, the methods disclosed herein can comprise calculating a first value of at least one parameter corresponding to a transcriptional efficiency of at least a portion of the nucleic acid associated with an analyte. In some embodiments, a transcriptional efficiency is correlated to a presence of at least one of the first binding site or a second binding site on the analyte. In some aspects, the methods disclosed herein can comprise calculating, with one or more computer processors, a first value of at least one parameter corresponding to a transcriptional efficiency of at least a portion of the nucleic acid associated with the analyte, and wherein the transcriptional efficiency is correlated to a presence of at least one of the first binding site or the second binding site on the analyte. In some aspects, the methods disclosed herein can comprise comparing a first value of at least one parameter to a reference value. In some aspects, the methods disclosed herein can comprise comparing, with the use of one or more computer processors, a first value of the at least one parameter to a reference value. In some aspects, the methods disclosed herein can comprise identifying a disease in a subject if a first value of the first parameter exceeds, is below or is the same as a reference value. In some aspects, the methods disclosed herein can comprise identifying, with the use of one or more computer processors, a disease in the subject if a first value of a first parameter exceeds a reference value. In some embodiments, a sample can be a biological sample. In some embodiments, a biological sample can be amniotic fluid, blood plasma, blood serum, breast milk, cells, cancer cells, tumor cells, cerebrospinal fluid, saliva, semen, synovial fluid, tears, tissue, cancer tissue, tumor tissue, urine, white blood cells, whole blood, and any fraction thereof. In some embodiments, a nucleic acid can be an intracellular nucleic acid. In some embodiments, a nucleic acid can be an extracellular nucleic acid. In some embodiments, a nucleic acid can be DNA. In some embodiments, a nucleic acid can be RNA. In some embodiments, a nucleic acid can be a hybrid of DNA and RNA. In some aspects, the methods disclosed herein comprise cross-linking a nucleic acid to an analyte using a cross-linking agent. In some aspects, the methods disclosed herein can comprise modifying a nucleic acid, wherein modifying can comprise generating a single stranded overhang at a first end of the nucleic acid or at a second end of the nucleic acid. In some embodiments, an extraction moiety can be biotin or a fragment thereof. In some embodiments, an extraction complex can comprise a polynucleotide linker. In some embodiments, an oligonucleotide can bind to a nucleic acid associated with an analyte. In some aspects, the methods disclosed herein can comprise dissociating a nucleic acid associated with an analyte from an extraction complex. In some embodiments, at least one of a first probe binds to a first binding site on an analyte or a second probe binds to a second binding site on an analyte or both. In some aspects, the methods disclosed herein can comprise attaching a first tag to a first end of a nucleic acid associated with an analyte and attaching a second tag to a second end of the nucleic acid associated with the analyte. In some aspects, the methods disclosed herein can comprise analyzing a nucleic acid, wherein analyzing the nucleic acid can comprise at least one of amplifying the nucleic acid or sequencing the nucleic acid. In some embodiments, sequencing can comprise multiplex sequencing. In some embodiments, amplifying comprises polymerase chain reaction.

In some aspects, this disclosure provides methods comprising associating a substrate to a first probe and a second probe. In some embodiments, a first probe can comprise a first tag comprising a first polynucleotide. In some embodiments, a second probe can comprise a second tag comprising a second polynucleotide. In some embodiments, a first probe can have an affinity to a first binding site on an analyte in a sample, and a second probe can have an affinity to a second binding site on the analyte. In some embodiments, a first tag can comprise a region for attaching to a first end of a nucleic acid associated with an analyte, and a second tag can comprise a region for attaching to a second end of the nucleic acid associated with the analyte. In some embodiments, a nucleic acid can be an intracellular nucleic acid. In some embodiments, a nucleic acid can be an extracellular nucleic acid. In some embodiments, a nucleic acid can be DNA. In some embodiments, a nucleic acid can be RNA. In some embodiments, a nucleic acid can be a hybrid of DNA and RNA. In some aspects, the methods disclosed herein can comprise cross-linking a nucleic acid to an analyte using a cross-linking agent. In some aspects, the methods disclosed herein can comprise modifying a nucleic acid, wherein modifying a nucleic acid can comprise generating a single stranded overhang at a first end of the nucleic acid or at the second end of the nucleic acid. In some aspects, the methods disclosed herein can comprise extracting a nucleic acid associated with an analyte from a sample. In some aspects, a nucleic acid associated with an analyte can be extracted from a sample by contacting the sample with an extraction complex. In some aspects, an extraction complex can comprise an extraction moiety and/or an oligonucleotide, wherein the extraction complex binds to the nucleic acid. In some embodiments, an extraction moiety can be biotin or a fragment thereof. In some embodiments, an extraction complex can comprise a polynucleotide linker. In some embodiments, an oligonucleotide can bind to a nucleic acid associated with an analyte. In some aspects, the methods disclosed herein can comprise dissociating a nucleic acid associated with an analyte from an extraction complex. In some embodiments, at least one of a first probe binds to a first binding site on an analyte or a second probe binds to a second binding site on the analyte. In some aspects, the methods disclosed herein can comprise attaching a first tag to a first end of a nucleic acid associated with an analyte and attaching a second tag to a second end of a nucleic acid associated with an analyte. In some aspects, the methods disclosed herein can comprise analyzing a nucleic acid, wherein analyzing a nucleic acid can comprise at least one of amplifying the nucleic acid or sequencing the nucleic acid. In some embodiments, sequencing can comprise multiplex sequencing. In some embodiments, amplifying can comprise polymerase chain reaction. In some embodiments of the methods disclosed herein, a substrate can be an array. In some embodiments, the methods disclosed herein can be performed in liquid phase or solid phase.

In some aspects, this disclosure provides kits comprising a targeting complex. In some embodiments, a targeting complex can comprise a first probe and a second probe. In some embodiments, a first probe and a second probe can be coupled to a solid substrate. In some embodiments, a first probe can comprise a first tag, and a second probe can comprise a second tag. In some embodiments, a first probe can have an affinity to a first binding site of a analyte and a second probe can have an affinity to a second binding site of the analyte. In some aspects, the kit comprises at least one buffer. In some embodiments, the kit comprises an instruction for using the kit.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

FIG. 1 generally depicts a method of analyzing a sample comprising a nucleic acid associated with an analyte by contacting the sample with a composition comprising a first probe and a second probe.

FIG. 2 depicts a tagged probe.

FIG. 3 depicts a method of preparing a tagged probe.

FIG. 4 depicts a method of detecting a protein dimer formation in cells

FIG. 5 depicts determination of optimal dilution of tagged probes to minimize ligation events not driven by protein-protein interactions.

FIG. 6 depicts an agarose gel analysis of GM12878 cell lysate dilution series incubated with tagged probes and subjected to ligation and PCR amplification.

FIG. 7 depicts an agarose gel analysis of GM12878, DH5a, and Hela cell lysate dilution series incubated with tagged probes and subjected to ligation and PCR amplification.

FIG. 8 depicts an agarose gel analysis of GM12878, DH5a, and Hela cell lysates incubated with tagged probes and subjected to ligation and PCR amplification.

FIG. 9 depicts an agarose gel analysis of GM12878, DH5a, and Hela cell lysates incubated with tagged probes and subjected to ligation and PCR amplification.

FIG. 10 depicts an agarose gel analysis of GM12878 cell lysate dilution series incubated with tagged probes and subjected to ligation and PCR amplification.

### DETAILED DESCRIPTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, i.e. the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed. The term “about” has the meaning as commonly understood by one of ordinary skill in the art. In some embodiments, the term “about” refers to ±10%. In some embodiments, the term “about” refers to ±5%.

The terms “attach”, “bind”, “couple”, and “link” are used interchangeably and refer to covalent interactions (e.g., by chemically coupling), or non-covalent interactions (e.g., ionic interactions, hydrophobic interactions, hydrogen bonds, hybridization, etc.).

The terms “specific”, “specifically”, or specificity” refer to the preferential recognition, contact, and formation of a stable complex between a first molecule and a second molecule compared to that of the first molecule with any one of a plurality of other molecules (e.g., substantially less to no recognition, contact, or formation of a stable complex between the first molecule and any one of the plurality of other molecules). For example, two molecules may be specifically attached, specifically bound, specifically coupled, or specifically linked. For example, specific hybridization between a first polynucleotide and a second polynucleotide can refer to the binding, duplexing, or hybridizing of the first polynucleotide preferentially to a particular nucleotide sequence of the second polynucleotide under stringent conditions. A sufficient number complementary base pairs in a polynucleotide sequence may be required to specifically hybridize with a target nucleic acid sequence. A high degree of complementarity may be needed for specificity and sensitivity involving hybridization, although it need not be 100%.

### Overview

Epigenetic modifications, such as the chemical modification of nucleic acids (e.g., DNA methylation) or the modification of an analyte associated with a nucleic acid (e.g., histones), can affect the transcriptional efficiency of a given gene, and even stop the gene from being transcribed altogether. In some instances, the outcome of transcription of a gene can depend on the presence of a particular combination of epigenetic modifications. However, current technology is only capable of surveying a single modification at a time. Many of the compositions and methods disclosed herein relate to the analysis of a nucleic acid associated with an analyte, wherein the nucleic acid or the analyte comprises at least two modifications. Whereas, in other embodiments, the nucleic acid or the analyte can comprise one or more modifications.

The present disclosure can enable a person having skill in the art to determine whether the transcriptional efficiency of a given gene is dependent on the presence of a particular modification or combination of modifications. Another advantage of the present disclosure is that the disclosure can enable a person having skill in the art to determine which modification or combinations of modifications exist at particular locations on a nucleic acid or analyte. Yet another advantage of the present disclosure is that the present disclosure can enable a person having skill in the art to correlate the modification patterns of a nucleic acid and/or an analyte in a sample from a subject with the presence or absence of a disease, Further, the present disclosure can enable a person having skill in the art to monitor a disease and/or the effect or effectiveness of a treatment based on the modification patterns of a nucleic acid and/or an analyte in a sample from a subject with the presence or absence of a disease,

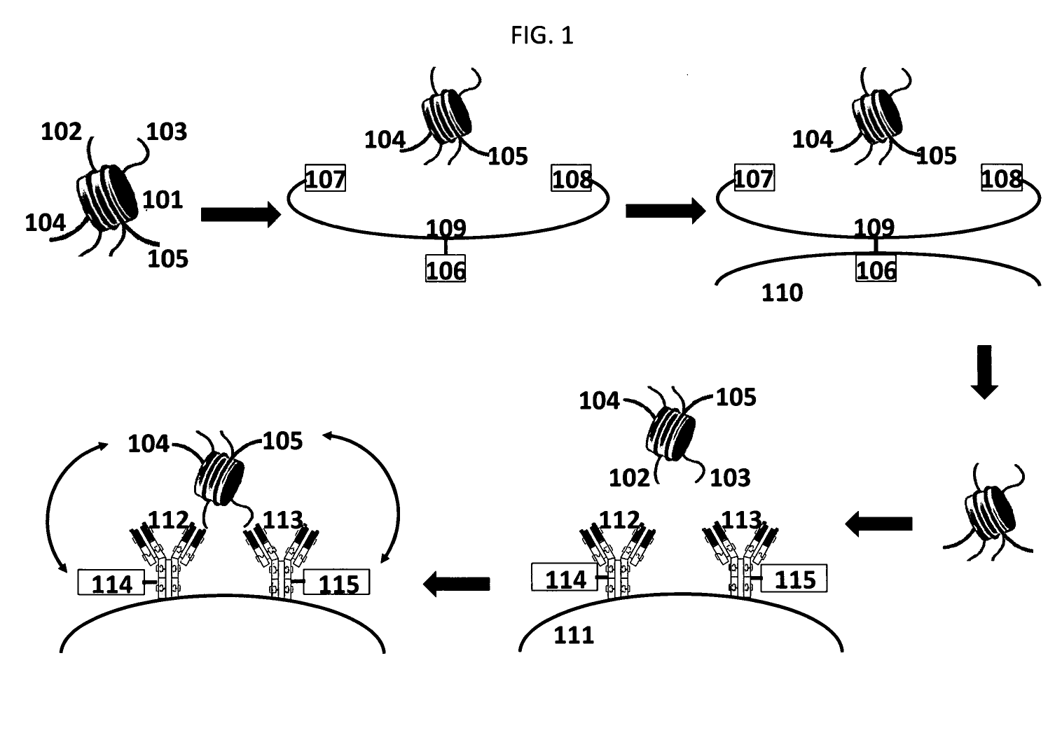

The compositions and methods disclosed herein generally relate to analyzing a nucleic acid associated with an analyte. FIG. 1 depicts a general schematic of some embodiments of the methods provided herein. The top left panel shows a sample comprising an analyte [101] (e.g., a histone octomer) comprising a first binding site [102] and a second binding site [103], and a nucleic acid with a first end [104] and a second end [105] associated with the analyte. The nucleic acid associated with the analyte can be contacted with an extraction complex comprising an extraction moiety comprising a first binding partner [106], a first oligonucleotide comprising an endonuclease recognition site [107], a second oligonucleotide comprising a second endonuclease recognition site [108], and a polynucleotide linker [109] linking the extraction moiety to the first oligonucleotide and the second oligonucleotide. Upon digestion of the first oligonucleotide and the first end of the nucleic acid with a first endonuclease, and digestion of the second oligonucleotide and the second end of the nucleic acid with a second endonuclease, the first oligonucleotide [107] can be ligated to the first end of the nucleic acid [104] using a first ligase, and the second oligonucleotide [108] can be ligated to the second end of the nucleic acid [105] using a second ligase. The extraction moiety can further comprise a second binding partner [110] that is a high affinity binding partner of the first binding partner [106], and is used to extract the nucleic acid associated with an analyte from the sample. After extracting the nucleic acid associated with the analyte using the extraction complex, the nucleic acid associated with the analyte can be dissociated from extraction complex. To selectively analyzing nucleic acids associated with an analyte comprising a first binding site [102] and a second binding site [103], the extracted sample can be contacted with a composition comprising a substrate [111]. A substrate can comprise a first probe [112] with an affinity to the first binding site [102], and a second probe [113] with an affinity to the second binding site [103]. The first probe can have a first tag [114] comprising a first cleavage site and a region for binding the first end of the nucleic acid [104]. The second probe can have second tag [115] comprising a second cleavage site and a region for binding the second end of the nucleic acid [105]. When each of the first probe and the second probe are bound to the first binding site and the second binding site on the analyte, the first probe [112] and the second probe [113] are in spatial proximity such that the first tag [114] can ligate to the first end of the nucleic acid [104] and the second tag [115] can ligate to the second end of the nucleic acid [105]. The first tag, the second tag, and the nucleic acid can be dissociated from the first probe and the second probe by cleaving the tag at the cleavage site. Following isolating the first tag, the second tag, and the nucleic acid from the analyte, the nucleic acid can be analyzed (e.g., amplified and/or sequenced).

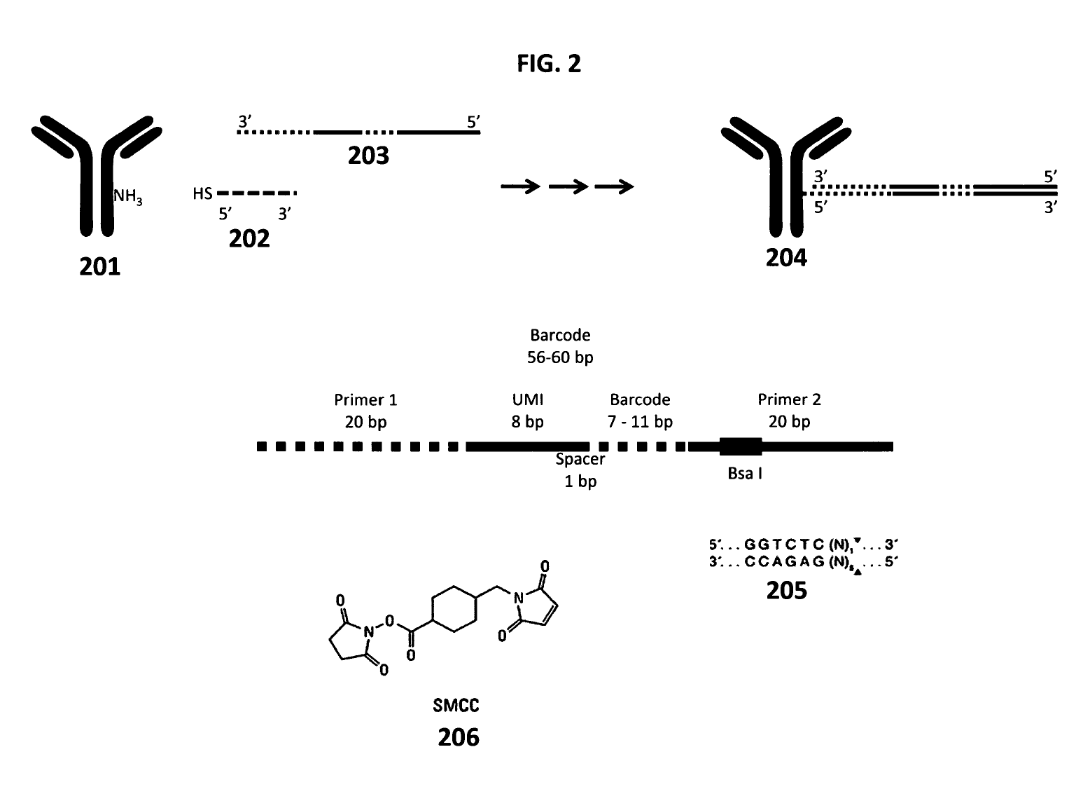

In some aspects, the compositions and methods disclosed herein generally relate to tagged probes. FIG. 2 depicts a general schematic of the preparation of a tagged probe. In an illustrative, non-limiting example, the probe is an antibody [201]. The probe can be combined with an oligonucleotide [202]. An oligonucleotide comprising a barcode [203] can hybridize or otherwise bind or associate with the oligonucleotide [202]. In some embodiments, an oligonucleotide comprising a barcode can comprise any one or more of the following: a primer 1 sequence, a unique molecular identifier (UMI) sequence, a barcode sequence, a restriction site (eg. BSA1), a spacer, and a primer 2 sequence. A primer, UMI, Barcode, restriction site, or a spacer disclosed herein can be 1, 2, 3, 4, 5, 6, 7, 8, 9, 10, 15, 20, 30, 40, 50, 100, 150, 200, 500, 1000 or more nucleotides. A sequence can comprise at least 1, 2, 3, 4, 5, 6, 7, 8, 9, 10, 15, 20, 30, 40, 50, 100, 150, 200, 500, 1000 unique primer sequence, UMI sequence, barcode sequence, restriction site sequence, or spacer sequence. In some instances one or more of a primer sequence, UMI sequence, barcode sequence, restriction site sequence, or spacer sequence can comprise the same nucleotide sequence.

A restriction site, for example BSA I restriction site can have a restriction sequence [205]. In some embodiments, In some aspects, a probe can be tagged or labeled by coupling or associating a 5′ sulfide of oligonucleotide [202] to an amine of a probe [201]. A oligonucleotide [203] can be hybridized to the oligonucleotide [202]. A 3′ end of oligonucleotide [202] can be extended using an enzyme and nucleotides [204].

In some embodiments, oligonucleotide [203] can be hybridized to oligonucleotide [202]. a 3′ end of oligonucleotide [202] can be extended using an enzyme and nucleotides, and a 5′ sulfide of oligonucleotide [202] can be coupled to an amine of probe [201] to form tagged probe [204].

In some embodiments, oligonucleotide [203] can be hybridized to oligonucleotide [202]. and a 5′ sulfide of oligonucleotide [202] can be coupled to an amine of probe [201] to form tagged probe [204]. In some embodiments, oligonucleotide [202] can comprise one or more of a primer 1, a UMI, a barcode, a spacer, a restriction site, a primer 2. A 5′ sulfide of oligonucleotide [202] can be coupled or be associated with an amine of probe [201] to form tagged probe.

In some embodiments, oligonucleotide [203] can be hybridized to oligonucleotide [202]. A 5′ sulfide of oligonucleotide [202] can be coupled to an amine of probe [201]. A 3′ end of oligonucleotide [202] can be extended using an enzyme and nucleotides to form a tagged probe [204].

In some embodiments, a sulfidryl group can be coupled to an amine group with a crosslinker [206]. In some embodiments, the cross-linker can comprise a succinimide moiety. In some embodiments, the crosslinker can comprise a maleimide moiety. In some embodiments, a crosslinker can comprise both a succinimide moiety and a maleimide moiety. In some embodiments, a cross-linker can be (succinimidyl 4-(N-maleimidomethyl)cyclohexane-1-carboxylate) (SMCC).

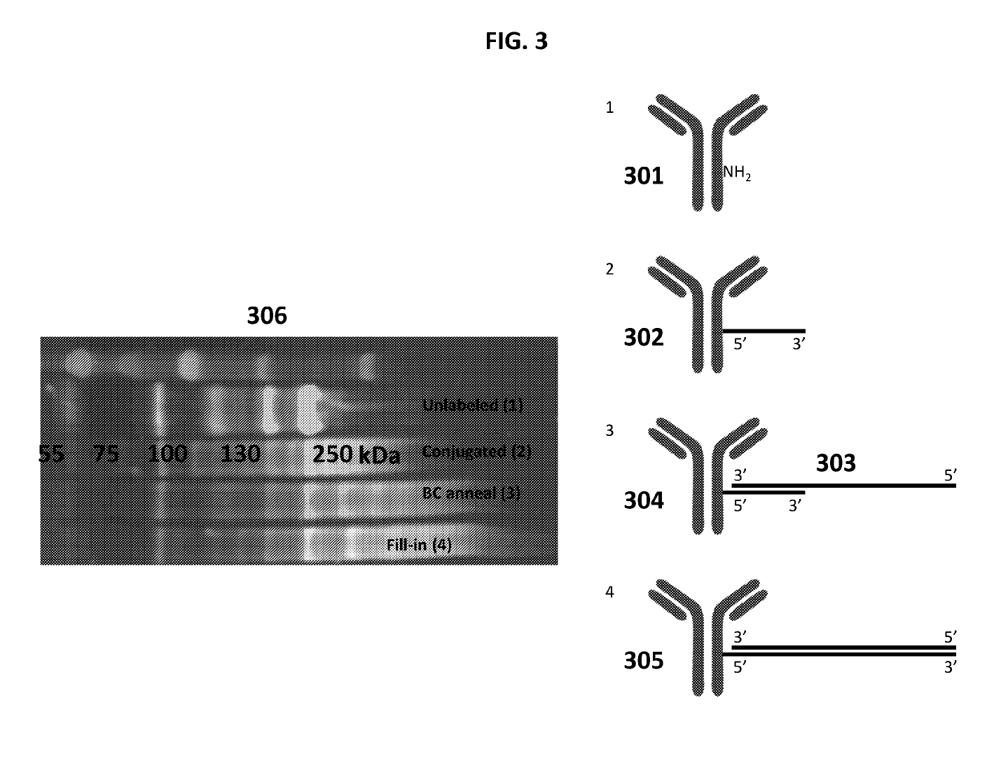

FIG. 3 depicts a general schematic of the preparation of a tagged probe, and a gel electrophoresis analysis of the same. In an illustrative, non-limiting example, a 5′ sulfide of an oligonucleotide can be coupled (conjugated) to an amine of unlabeled probe [301]. An oligonucleotide can be hybridized (annealed) to a barcode oligonucleotide [303] to form an annealed probe [304]. A 3′ end of the oligonucleotide can be extended (fill-in) using an enzyme and nucleotides to form a tagged probe [305]. The left panel of FIG. 3 [306] depicts a gel electrophoresis (3-8% PageTris-Acetate) of each of the steps of formation of a tagged probe identified with green-αlgG and red-dCTP.

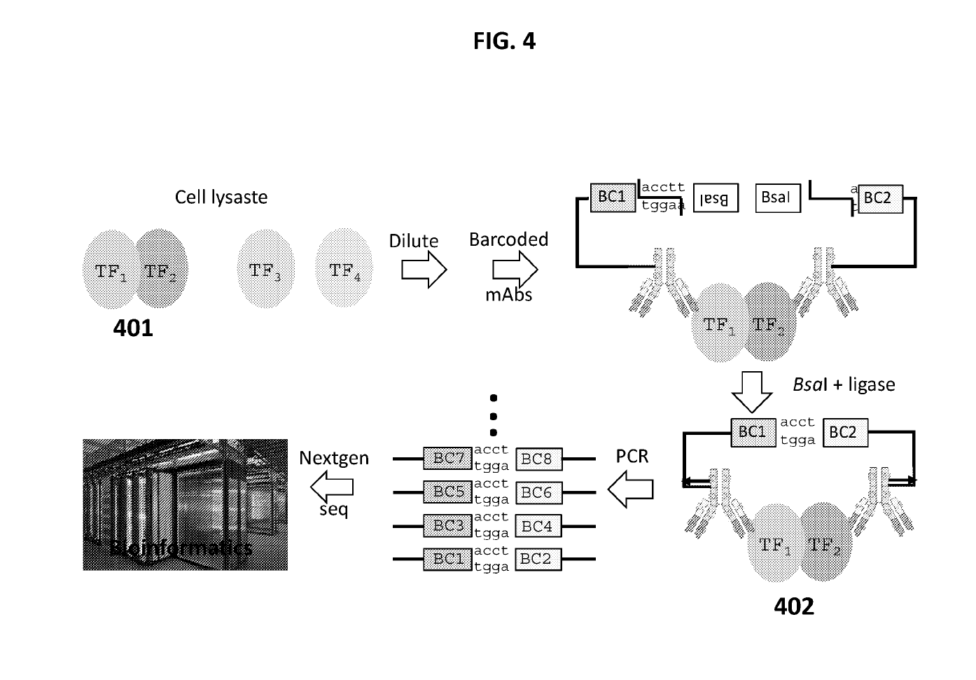

FIG. 4 depicts a general schematic of a method for detecting a protein dimer formation in a cell. In an illustrative, non-limiting example, a cell lysate contains proteins including the transcription factors TF₁, TF₂, TF₃, and TF₄. In some instances, TF₁ and TF₂ together form a dimer [401], while TF₃ and TF₄ do not form a dimer. The cell lysate can be diluted, and a first probe and a second probe can be added. In the illustrated instance, the first probe can be an antibody that has binding specificity for TF₁, and comprises a tag comprising a barcode sequence BC1 and a restriction site (e.g Bsa1). In the illustrated instance, the second probe can be an antibody that has binding specificity for TF₂, and comprises a tag comprising a barcode sequence BC2 and a restriction site (e.g Bsa1). After binding of the first probe and the second probe to TF₁ and TF₂, respectively, the mixture can be treated with a restriction enzyme (eg.) Bsa1 and a ligase. In an embodiment, Bsa1 can cleave the restriction site on each of the first and second probe. Because TF₁ and TF₂ form a dimer, the respective tags of the first probe and the second probe are in proximity to each other, and the ligase ligates the ends of the tags together to form a ligated dimer [402]. PCR amplification of the ligated nucleotide sequence can produce a PCR product containing a BC1-BC2 sequence, indicating the formation of a dimer between the analytes bound by the first probe and second probe (i.e. TF₁ and TF₂). In some embodiments, PCR and next generation sequencing can determine formation of multiple dimers simultaneously. In some embodiments, bioinformatics can be employed to analyze the results of next generation sequencing. In other embodiments, the method disclosed can be used to identify a presence of TF₁ and/or TF₂ or a lack thereof.

### Compositions

The compositions disclosed herein are generally useful for analyzing nucleic acids (e.g., genomic DNA). A person of skill in the art will appreciate that a nucleic acid can generally refer to a substance whose molecules consist of many nucleotides linked in a long chain. Non-limiting examples of the nucleic acid include an artificial nucleic acid analog (e.g., a peptide nucleic acid, a morpholino oligomer, a locked nucleic acid, a glycol nucleic acid, or a threose nucleic acid), chromatin, niRNA, cDNA, DNA, single stranded DNA, double stranded DNA, genomic DNA, plasmid DNA, or RNA. In some embodiments, nucleic acid can be double stranded or single stranded. In some embodiments, a sample can comprise a nucleic acid, and the nucleic acid can be intracellular. In some embodiments, a sample can comprise a nucleic acid, and the nucleic acid can be extracellular (e.g., cell-free). Cell-free nucleic acids can be cell-free DNA, cell-free RNA (e.g., cell-free mRNA, cell-free miRNA, cell-free siRNA), or any combination thereof. In certain cases, cell-free nucleic acids can be pathogen nucleic acids, e.g., nucleic acids from pathogens. Cell-free nucleic acids may be circulating nucleic acids, e.g., circulating tumor DNA or circulating fetal DNA. As used herein, the term “cell-free” refers to the condition of the nucleic acid as it appeared in the body before the sample is obtained from the body. For example, circulating cell-free nucleic acids in a sample may have originated as cell-free nucleic acids circulating in the bloodstream of the human body. In contrast, nucleic acids that are extracted from a solid tissue, such as a biopsy, are generally not considered to be “cell-free.”

In some embodiments, a sample can comprise a nucleic acid (e.g. chromatin), and the nucleic acid can be fragmented.

### Analyte

In some aspects, the compositions disclosed herein are useful for analyzing nucleic acids associated with an analyte. In some embodiments, an analyte can comprise a biological molecule or a non-biological molecule. In some embodiments, an analyte can comprise a biological molecule or a non-biological molecule, and the biological or non-biological molecule can be associated with a nucleic acid. In some embodiments, a biological molecule or non-biological molecule can be a naturally occurring molecule or an artificial molecule. Non-limiting examples of a biological molecule include a protein, a carbohydrate, a lipid, or a nucleic acid. Non-limiting examples of an analyte include a bead, a carbohydrate, a DNA-binding protein, a histone, a lipid, a nuclease, a nucleosome, a polymerase, a protein, a peptide, a cell, a cytokine, organelles, a transcription factor, or any combination thereof. The analyte can comprise multiple subunits. In some embodiments, an analyte can comprise multiple subunits, and the subunits can be the same. In some embodiments, an analyte can comprise multiple different subunits. In some embodiments, an analyte can comprise multiple subunits, and at least two of the subunits can be different.

In some embodiments the analyte can comprises a histone, and the histone can be a linker histone. Non-limiting examples of a linker histone include but is not limited to histone H1, histone H1F, histone H1F0, histone H1FNT, histone H1FOO, histone H1FX, histone H1H1, histone HIST1H1A, histone HIST1H1B, histone HIST1H1C, histone HIST1H1D, histone HIST1H1E, histone HIST1H1T, or any combination thereof. In some embodiments disclosed herein, the analyte can comprise a histone, and the histone can be a core histone. Non-limiting examples of a core histone include histone H2A, histone H2AF, histone H2AFB1, histone H2AFB2, histone H2AFB3, histone H2AFJ, histone H2AFV, histone H2AFX, histone H2AFY, histone H2AFY2, histone H2AFZ, histone H2A1, histone HIST1H2AA, histone HIST1H2AB, histone HIST1H2AC, histone HIST1H2AD, histone HIST1H2AE, histone HIST1H2AG, histone HIST1H2AI, histone HIST1H2AJ, histone HIST1H2AK, histone HIST1H2AL, histone HIST1H2AM, histone H2A2, histone HIST2H2AA3, histone HIST2H2AC, histone H2B, histone H2BF, histone H2BFM, histone H2BFS, histone H2BFWT, histone H2B1, histone HIST1H2BA, histone HIST1H2BB, histone HIST1H2BC, histone HIST1H2BD, histone HIST1H2BE, histone HIST1H2BF, histone HIST1H2BG, histone HIST1H2BH, histone HIST1H2BI, histone HIST1H2BJ, histone HIST1H2BK, histone HIST1H2BL, histone HIST1H2BM, histone HIST1H2BN, histone HIST1H2BO, histone H2B2, histone HIST2H2BE, histone H3, histone H3A1, histone HIST1H3A, histone HIST1H3B, histone HIST1H3C, histone HIST1H3D, histone HIST1H3E, histone HIST1H3F, histone HIST1H3G, histone HIST1H3H, histone HIST1H3I, histone HIST1H3J, histone H3A2, histone HIST2H3C, histone H3A3, histone HIST3H3, histone H4, histone H41, histone HIST1H4A, HIST1H4B, HIST1H4C, HIST1H4D, HIST1H4E, HIST1H4F, HIST1H4G, HIST1H4H, histone HIST1H4I, histone HIST1H4J, histone HIST1H4K, histone HIST1H4L, histone H44, histone HIST4H4, or any combination thereof. In some embodiments, an analyte can comprise a linker histone and a core histone. In some embodiments, an analyte can comprise a monomer. In some embodiments, an analyte can comprise an octomer. In some embodiments, an analyte can comprise a dimer, trimer, tetramer, pentamer, hexamer, heptamer, nonamer, or decamer. In some embodiments, an analyte can comprise greater than about ten subunits. In some embodiments, an analyte can comprise a polymer. In some embodiments, an analyte can comprise a plurality of proteins. For example, in some embodiments disclosed herein, the analyte can comprise a histone octomer (e.g., an eight protein complex comprising two copies of each of four core histone proteins).

In one aspect, provided herein are compositions comprising a first probe, wherein the first probe comprises a first tag comprising a polynucleotide comprising a region for attaching to a first end of a nucleic acid; and a second probe, wherein the second probe comprises a second tag comprising a polynucleotide comprising a region for attaching to a second end of the nucleic acid, wherein the first probe has an affinity to a first binding site on an analyte and the second probe has an affinity to a second binding site on the analyte, wherein the first probe and the second probe are in spatial proximity, and (i) wherein the first probe is associated with a substrate; (ii) wherein the second probe is associated with the substrate; (iii) wherein the first probe is associated with the substrate and wherein the second probe is associated with the substrate; (iv) wherein the first tag is double stranded where associated with the first probe; (v) wherein the second tag is double stranded where associated with the second probe; (vi) wherein the first tag is double stranded where associated with the first probe and wherein the second tag is double stranded where associated with the second probe; or (vii) one of (i), (ii), or (iii) and one of (iv), (v) or (vi). In some embodiments, the first probe can be associated with a solid substrate. In some embodiments, the second probe can be associated with the solid substrate.

### Analyte Coupled to a Substrate

An analyte can be coupled to a solid support. For example, an analyte can be immobilized on a solid substrate. An analyte can be coupled to the solid support through covalent or non-covalent interactions. For example, an analyte can be coupled to the solid support non-covalently through hydrophobic bonding, hydrogen bonding, Van der Waals interactions, ionic bonding, etc. In some instances, an analyte is coupled reversibly. In some instances, an analyte is coupled irreversibly.

An analyte can be coupled a solid support through a functional group (e.g., a reactive group). An analyte can comprise any suitable functional group for coupling to a solid support. For example, a surface of a solid support can be coated with a functional group and an analyte can be attached to the solid support through the functional group. For example, a solid support can be coated with a first functional group and an analyte comprising a second functional group can be attached to the solid support by binding or reacting the first and second functional groups. For example, a surface of a solid support can be coated with streptavidin and a biotinylated analyte can be attached thereto.

An analyte or functional group for attachment of an analyte can be deposited on a solid surface (e.g., an array or bead) by any suitable technique. Examples of solid surface materials and corresponding functional groups include gold, silver, copper, cadmium, zinc, palladium, platinum, mercury, lead, iron, chromium, manganese, tungsten, and any alloys thereof. Exemplary functional groups of solid surfaces include sulfur-containing functional groups such as thiols, sulfides, disulfides (e.g., —SR or —SSR where R is H, alkyl, or aryl), and the like; doped or undoped silicon with silanes and chlorosilanes (e.g., —SiR2Cl where R is H, alkyl, or aryl); metal oxides (e.g., silica, alumina, quartz, glass, and the like) with carboxylic acids; platinum and palladium with nitrites and isonitriles; copper with hydroxamic acids; benzophenones; acid chlorides; anhydrides; epoxides; sulfonyl groups; phosphoryl groups; hydroxyl groups; phosphonates; phosphonic acids; amino acid groups; amides; and the like (See, e.g., U.S. Pat. No. 6,413,587). An analyte can optionally be coupled to a solid support through one or more bifunctional linkers (e.g., the linkers comprising one functional group capable of forming a linkage with a solid substrate and another functional group capable of forming a linkage with another linker molecule or analyte). Depending on the particular application, linkers may be long or short, flexible or rigid, charged or uncharged, and/or hydrophobic or hydrophilic.

A substrate can be coated for a variety of reasons, for example, to alter the hydrophilic properties of the substrate (e.g., surface wetting), to enhance or prevent binding to a ligand or binding partner, or to shield the negative space on a substrate from subsequent coatings/treatments (e.g., for micropatterning). In some embodiments, a substrate can be completely coated. In some embodiments, a substrate can be partially coated. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the substrate is coated. In some embodiments, a substrate can be coated with one or more chemical compounds (e.g., an iodoacetyl functional group). In some embodiments, at least a portion of the substrate is coated with a polymer. Non limiting examples of polymers that can be used to coat a substrate include polyethylene glycol, polymethacrylate, polymethylmethacrylate, polyethylenimine, polyvinyl alcohol, polyvinyl acetate, polystyrene, polyglutaraldehyde, polyacrylamide, agarose, chitosan, alginate, or a combination thereof. In some embodiments comprising a substrate contacted with a polymer or a first binding partner, the analyte can be conjugated to a substrate chemically or enzymatically. For example, an analyte comprising an antibody can be chemically conjugated to a substrate which has been at least 60% coated with polyethylene glycol. In some embodiments, at least a portion of a substrate can be coated with a first binding partner which has an affinity for a second binding partner. Non limiting examples of the first binding partner or second binding partner include antibody, immunoglobulin-binding protein, Protein A, Protein G, Protein A/G, calmodulin, glutathione, glutathione S-transferase (GST), streptavidin, avidin, maltose-binding protein, a His tag, or a combination thereof. In one example, a substrate can be a spherical substrate, and the spherical substrate can be coated with Protein G. In some embodiments, the substrate can be coated to enable or promote association between the analyte and the substrate. For example, a substrate can be coated with a first binding partner which has an affinity for a second binding partner, wherein the analyte comprises the second binding partner, thereby enabling association between the analyte and the substrate. Any of the embodiments disclosed herein can comprise a substrate which is at least partially coated with both of a polymer and a first binding partner which has an affinity for a second binding partner. A substrate can be coated using any method known in the art. In some embodiments, coating the substrate can comprise physical modification, chemical modification, photochemical modification, graft formation, plasma treatment, covalent immobilization, the wet chemical method, Staudinger ligation, alkali hydrolysis, or a combination thereof. For example, a spherical substrate can be coated with streptavidin by covalent immobilization.

Non limiting examples of a binding partner, a first binding partner or a second binding partner include antibody, immunoglobulin-binding protein, Protein A, Protein G, Protein A/G, calmodulin, glutathione, glutathione S-transferase (GST), streptavidin, avidin, maltose-binding protein, a His tag, or a combination thereof. In one example, the analyte can comprise a polymer, and the polymer can be covalently immobilized directly onto the substrate. In another example, a substrate can be coated with the first binding partner GST, and the analyte can comprise the second binding partner glutathione.

### Probes

In some embodiments, the compositions can comprise a probe (e.g., a first probe and/or a second probe). In some embodiments a probe can comprise an antibody or fragment thereof. In some embodiments, a probe can comprise a compound capable of identifying and/or targeting an antigen (e.g., a probe can comprise an antibody or an antibody mimetic). Non-limiting examples of a probe can include an antibody, affibodies, affilins, affimers, affitins, alphabodies, anticalins, aptamers, avimers, DARPins, fynomers, Kunitz domain peptides, transcription factors, monobodies, or a fragment and/or combination thereof. In some embodiments, a probe can comprise a nucleic acid (e.g., an aptamer). Aptamers can generally refer to an engineered nucleic acid that has been selected for its ability to bind to various molecular targets such as small molecules, proteins, nucleic acids, and even cells, tissues and organisms. In some embodiments, the probe can comprise an antibody, and the antibody can comprise an IgA isotype antibody, an IgD isotype antibody, an IgE isotype antibody, an IgG isotype antibody, an IgM isotype antibody, an IgW isotype antibody, an IgY isotype antibody, or a fragment and/or combination thereof. In some embodiments, the probe can comprise an antibody, and the antibody can be monomeric. In some embodiments, the probe can comprise an antibody, and the antibody can be dimeric. In some embodiments, the probe can comprise an antibody, and the antibody can be homodimeric. In some embodiments, the probe can comprise an antibody, and the antibody can be bispecific. In some embodiments, a probe can comprise an antibody, and the antibody can be isolated and/or purified from a hybridoma. Generally, a hybridoma can comprise any hybrid cell line produced by the fusion of a white blood cell (e.g., a B cell) and an immortalized B cell cancer cell (e.g., a myeloma), wherein the hybrid cell line has both the antibody-producing ability of the B-cell and the exaggerated longevity and reproductively of the immortalized B cell cancer cell. In some embodiments, the probe comprises an antibody, and the antibody is a monoclonal antibody, a recombinant antibody, a polyclonal antibody, a chimeric antibody, a humanized antibody, a bispecific antibody, or a combination or a fragment thereof. For example, a probe can comprise a monoclonal antibody. In another example, a probe can comprise a fragment of a polyclonal antibody.

In some embodiments, the compositions disclosed herein can comprise a probe (e.g., a first probe and/or a second probe), and the probe comprises at least one tag (e.g., a first tag or a second tag). In some embodiments, the tag can comprise DNA, RNA, or a hybrid of DNA and RNA. In some embodiments the tag can be single stranded, double stranded, or a combination thereof. For example, a probe can comprise a tag, and the tag can be double stranded. In another example, a probe can comprise a tag, and the tag can be double stranded where associated with the probe. In yet another example, a probe can comprise a tag, the tag can be double stranded at a first end of the tag where associated with the probe, and single stranded (e.g., comprising a sticky end or overhang) at a second end of the tag.

In some embodiments, the compositions disclosed herein can comprise a probe (e.g., a first probe and/or a second probe), and the probe comprises at least one tag (e.g., a first tag or a second tag). In some embodiments, the tag can comprise promoter regions, barcodes, restriction sites, cleavage sites, endonuclease recognition sites, primer binding sites, selectable markers, unique identification sequences, resistance genes, linker sequences, or any combination thereof. In some embodiments, the tag (e.g., a first tag or a second tag) can comprise a cleavage site (e.g., a first cleavage site or a second cleavage site).

### Cleavage Site

A cleavage site can generally refer to a specific peptide or nucleotide sequences at which site-specific molecules (e.g., proteases, endonucleases, or enzymes) can cut the protein or polynucleotide. In some embodiments, an oligonucleotide, a polynucleotide, a nucleic acid or the like can comprise a restriction site. In one example, a probe can comprise a tag, and the tag can comprise a cleavage site, wherein cleaving the tag at the cleavage site releases the tag from the probe. In some embodiments, the cleavage site can comprise at least one endonuclease recognition site. In some embodiments, the endonuclease recognition site can comprise a Type I endonuclease recognition site, a Type II endonuclease recognition site, a Type III endonuclease recognition site, a Type IV endonuclease recognition site, or a Type V endonuclease recognition site. Non-limiting examples of endonuclease recognition sites include an AatII recognition site, an Acc65I recognition site, an AccI recognition site, an AclI recognition site, an AatII recognition site, an Acc65I recognition site, an AccI recognition site, an AclI recognition site, an AfeI recognition site, an AflII recognition site, an AgeI recognition site, an ApaI recognition site, an ApaLI recognition site, an ApoI recognition site, an AscI recognition site, an AseI recognition site, an AsiSI recognition site, an AvrII recognition site, a BamHI recognition site, a BclI recognition site, a BglII recognition site, a Bme1580I recognition site, a BmtI recognition site, a BsaI recognition site, a BsaHI recognition site, a BsiEI recognition site, a BsiWI recognition site, a BspEI recognition site, a BspHI recognition site, a BsrGI recognition site, a BssHII recognition site, a BstBI recognition site, a BstZ17I recognition site, a BtgI recognition site, a ClaI recognition site, a DraI recognition site, an EaeI recognition site, an EagI recognition site, an EcoRI recognition site, an EcoRV recognition site, an FseI recognition site, an FspI recognition site, an HaeII recognition site, an HincII recognition site, a HindIII recognition site, an HpaI recognition site, a KasI recognition site, a KpnI recognition site, an MfeI recognition site, an MluI recognition site, an MscI recognition site, an MspA1I recognition site, an MfeI recognition site, an MluI recognition site, an MscI recognition site, an MspA1I recognition site, an NaeI recognition site, a NarI recognition site, an NcoI recognition site, an NdeI recognition site, an NgoMIV recognition site, an NheI recognition site, a NotI recognition site, an NruI recognition site, an NsiI recognition site, an NspI recognition site, a PacI recognition site, a PciI recognition site, a PmeI recognition site, a PmlI recognition site, a PsiI recognition site, a PspOMI recognition site, a PstI recognition site, a PvuI recognition site, a PvuII recognition site, a SacI recognition site, a SacII recognition site, a SalI recognition site, an SbfI recognition site, an ScaI recognition site, an SfcI recognition site, an SfoI recognition site, an SgrAI recognition site, an SmaI recognition site, an SmlI recognition site, an SnaBI recognition site, an SpeI recognition site, an SphI recognition site, an SspI recognition site, an StuI recognition site, an SwaI recognition site, an XbaI recognition site, an XhoI recognition site, and an XmaI recognition site. In a particular example, the cleavage site can comprise BsaI endonuclease recognition site.

### Tag

In some embodiments, the compositions disclosed herein can comprise a probe (e.g., a first probe and/or a second probe), and the probe comprises at least one tag (e.g., a first tag or a second tag). In some embodiments, a tag can comprise a polynucleotide. Generally, a polynucleotide can refer to a linear polymer whose molecule is composed of many nucleotide units. A polynucleotide can comprise any number of polynucleotides. In some embodiments, a polynucleotide can comprise less than about 10, 15, 20, 25, 30, 40, 50, 100 nucleotides. In some embodiments, a polynucleotide can comprise at least about 10, 50, 70, 100, 500, 1000, 2000 nucleotides. In some embodiments, a polynucleotide can comprise between about 5 and about 50 nucleotides. In some embodiments, a polynucleotide can comprise between about 50 and about 100 nucleotides. In some embodiments, a polynucleotide can comprise between about 100 and about 150 nucleotides. In any of the embodiments disclosed herein, a tag can comprise DNA, RNA, or a hybrid of DNA and RNA. In some embodiments, a polynucleotide can be single stranded. In some embodiments, a polynucleotide can be double stranded. In some embodiments, a polynucleotide as disclosed in any of the embodiments herein can comprise promoter regions, restriction sites, cleavage sites, endonuclease recognition sites, primer binding sites, selectable markers, unique identification sequences, resistance genes, linker sequences, spacers or any combination thereof. In some aspects, these sites can be useful for enzymatic digestion, amplification, sequencing, targeted binding, purification, or any combination thereof. In some embodiments, a polynucleotide can comprise a region for attaching to a first end or a second end of a nucleic acid. In some embodiments, a region for attaching to a first end or a second end of a nucleic acid can be at the end of a polynucleotide. In some embodiments, a polynucleotide can readily bind to the nucleic acid (e.g., the polynucleotide comprises a sticky end or nucleotide overhang). For example, a polynucleotide can comprise an overhang at a first end of the polynucleotide. Generally, a sticky end or overhang can refer to a series of unpaired nucleotides at the end of a polynucleotide. In some embodiments, a polynucleotide can comprise a single stranded overhang at one or more ends of the polynucleotide. In some embodiments, the overhang can occur on the 3′ end of a polynucleotide. In some embodiments, the overhang can occur on the 5′ end of a polynucleotide. An overhang can comprise any number of nucleotides. For example, an overhang can comprise at last about 1, 2, 3, 4, 5, 6, 7, 8, 9, 10, 15, 20, 30, 40, 50 or more nucleotides. In some embodiments, the region for attaching to a first end or a second end of a nucleic acid can be within a polynucleotide. In some embodiments, a polynucleotide can require modification prior to binding to a nucleic acid (e.g., the polynucleotide can be digested with an endonuclease). In some embodiments, modification of the polynucleotide can generate a nucleotide overhang, and an overhang can comprise any number of nucleotides. In some embodiments, an overhang can comprise at last about 1, 2, 3, 4, 5, 6, 7, 8, 9, 10, 15, 20, 30, 40, 50 or more nucleotides. In one example, the polynucleotide can comprise a restriction site. In some embodiments, digesting a polynucleotide at a restriction site with a restriction enzyme (e.g., NotI) can produce a nucleotide overhang (e.g., a 4 nucleotide overhang). In some embodiments, modifying can comprise generating a blunt end at one or more ends of a polynucleotide. Generally, a blunt end can refer to a double stranded polynucleotide wherein both strands terminate in a base pair. In one example, the polynucleotide can comprise a restriction site, wherein digesting the polynucleotide at the restriction site with a restriction enzyme (e.g., BsaI) produces a blunt end.

In some embodiments, the compositions disclosed herein can comprise a probe (e.g., a first probe and/or a second probe), and the probe comprises at least one tag (e.g., a first tag or a second tag). In some embodiments, the tag can comprise a barcode. A barcode sequence can generally refer to a series of nucleotides that allows for the unique identification of the corresponding probe. A barcode sequence can have any number of nucleotides. A barcode can comprise any number of polynucleotides. In some embodiments, a barcode can comprise less than about 10 nucleotides. In some embodiments, a barcode can comprise at least about 10 nucleotides. In some embodiments, a barcode can comprise at least about 20 nucleotides. In some embodiments, a barcode can comprise at least about 30 nucleotides. In some embodiments, a barcode can comprise at least about 40 nucleotides. In some embodiments, a barcode can comprise at least about 50 nucleotides. In some embodiments, a barcode can comprise at least about 75 nucleotides. In some embodiments, a barcode can comprise at least about 100 nucleotides. In some embodiments, a barcode can comprise at least about 500 nucleotides. In some embodiments, a barcode can comprise at least about 1000 nucleotides. In some embodiments, a barcode can comprise between about 5 and about 50 nucleotides. In some embodiments, a barcode can comprise between about 50 and about 100 nucleotides. In some embodiments, a barcode can comprise between about 100 and about 150 nucleotides. For example, a probe can comprise a tag, and the tag can comprise a 20 nucleotide barcode. In another example, a barcode sequence can comprise between about 50 nucleotides and about 75 nucleotides.

In some embodiments, the compositions disclosed herein can comprise a probe (e.g., a first probe and/or a second probe), and the probe can comprises at least one tag (e.g., a first tag or a second tag). In some embodiments, the tag can comprise a primer binding site. Generally, a primer binding site is a region of a nucleic acid where a single-stranded oligonucleotide binds to initiate replication. In some embodiments comprising a double stranded nucleic acid, the primer binding site can be on one of two complementary strands (e.g., the strand to be copied). A primer binding site can comprise any number of nucleotides. In some embodiments, the primer binding site can comprise about 1 to 50 nucleotides. In some embodiments, the primer binding site can comprise 18 to 22 nucleotides. In some embodiments, the GC content (e.g., the number of guanine and cytosine nucleotides as a percentage of the total number of nucleotides in the primer binding site) can be about 30% to 70%. In some embodiments, the GC content can be less than 40%. In some embodiments, the GC content can be greater than 60%.

In some embodiments, the compositions disclosed herein can comprise a probe (e.g., a first probe and/or a second probe), and the probe can comprises at least one tag (e.g., a first tag or a second tag). In some embodiments, a tag can comprise a cleavage site, a polynucleotide and a barcode. In any of the embodiments disclosed herein, a cleavage site, a polynucleotide, and a barcode can appear in any order and/or combination on in a tag. In one embodiment, from a first end of a tag associated with a probe to a second end of the tag, a tag can comprise a cleavage site, a barcode, and a polynucleotide. In some embodiments, the cleavage site can be positioned relative to a barcode and a polynucleotide such that, after a polynucleotide ligates to a nucleic acid, upon cleavage at the cleavage site, the barcode, polynucleotide and nucleic acid are separated from the probe. In some embodiments, from a first end of a tag associated with a probe to a second end of the tag, a tag can comprise a barcode and a cleavage site. In some embodiments, from a first end of a tag associated with a probe to a second end of the tag, a tag can comprise a barcode and a polynucleotide comprising a cleavage site.

### Modified Residue

In some embodiments, the compositions disclosed herein can comprise a probe (e.g., a first probe and/or a second probe), and the probe has an affinity to a binding site on an analyte. A binding site on an analyte can generally refer to a region on the analyte to which a probe (e.g., an antibody) can associate. In some embodiments, the binding site can comprise an antigen. In some embodiments, the binding site can comprise an antigen, and the antigen can comprise a modified residue. Non-limiting examples of a modified residue include acetylation, acylation, adenylylation, amidation, arginylation, biotinylation, carbamylation, carbonylation, carboxylation, citrullination, eliminylation, farnesylation, formylation, glycation, glycosylation, glypiation, hydroxylation, imination, isoprenylation, lipidation, lipoylation, malonylation, methylation, myristoylation, Neddylation, nitrosylation, oxidation, palmitoylation, pegylation, phophopantetheinylation, phosphorylation, polyglutamylation, prenylation, Pupylation, succinylation, sulfation, sumoylation, ubiquitylation, and/or any combination thereof. In some embodiments, the residue modification can comprise the absence of a residue or the absence of a fragment of a residue. In one example, a binding site can comprise an antigen, and the antigen can comprise a residue where a methyl group is absent or has been removed. In some embodiments, the modified residue can comprise de-acetylation, de-acylation, de-adenylylation, de-amidation, de-arginylation, de-biotinylation, de-carbamylation, de-carbonylation, de-carboxylation, de-citrullination, de-eliminylation, de-farnesylation, de-formylation, de-glycation, de-glycosylation, de-glypiation, de-hydroxylation, de-imination, de-isoprenylation, de-lipidation, de-lipoylation, de-malonylation, de-methylation, de-myristoylation, de-Neddylation, de-nitrosylation, de-oxidation, de-palmitoylation, de-pegylation, de-phophopantetheinylation, de-phosphorylation, de-polyglutamylation, de-prenylation, de-Pupylation, de-succinylation, de-sulfation, de-sumoylation, de-ubiquitylation, and/or any combination thereof.

In some embodiments comprising a first probe and/or a second probe, the first probe can have an affinity to a first binding site on an analyte and the second probe can have an affinity to a second binding site on the analyte. In some embodiments, a first binding site can comprise the same modified residue as the second binding site. For example, in an embodiment comprising a first probe and a second probe, the first probe can have an affinity to a first methylation site on an analyte and the second probe can have an affinity to a second methylation site on the analyte. In some embodiments, the first binding site can comprise a different modified residue than the second binding site. For example, in an embodiment comprising a first probe and a second probe, the first probe can have an affinity to a methylation site on an analyte and the second probe can have an affinity to an acetylation site on the analyte. Generally, the embodiments disclosed herein can comprise a first probe that has an affinity to a first binding site on an analyte and a second probe that has an affinity to a second binding site on the analyte, however a probe can bind to more than one binding site. In one example, a probe can comprise an antibody, wherein the antibody is a bispecific antibody capable of binding to two distinct binding sites on the analyte.

### Complex

In some aspects of the embodiments disclosed herein comprising a first probe and/or a second probe, the first probe and the second probe can be in spatial proximity. In some embodiments, the first probe and the second probe can be in spatial proximity to form a complex with the analyte. In some aspects, spatial proximity can generally refer to a distance wherein both a first probe and a second probe are able to form a complex with an analyte. In some instances, a complex can comprise a probe associating with an analyte. In some instances, a complex can comprise two probes associating with an analyte. In some instances, a complex can comprise at least two probes associating with an analyte. In some instances, a complex comprises a probe associating with a histone modification. In some instances, a complex comprises two probes, each associating with a separate histone modification. In some instances, a complex comprises at least two probes, wherein each probe associates with a separate histone modification. In some instances, a complex comprises a probe associating with a post-translational modification. In some instances, a complex comprises two probes, each associating with a separate post-translational modification. In some instances, a complex comprises at least two probes, wherein each probe associates with a separate post translational modification.

In some instances, a complex can comprise a probe comprising a tag, wherein the tag associates (e.g., by ligation) with a nucleic acid. In some instances, a complex can comprise two probes each comprising a tag, wherein at least one tag associates (e.g., by ligation) with a nucleic acid. In some instances, a complex comprises two probes each comprising a tag, wherein the first tag associates (e.g., by ligation) with a first end of a nucleic acid and the second tag associated with a second end of the nucleic acid. In some instances, a complex can comprise a probe comprising a tag, wherein the tag associates (e.g., by ligation) with a nucleic acid, and the nucleic acid is associated with an analyte. In some instances, a complex can comprise two probes each comprising a tag, wherein at least one tag associates (e.g., by ligation) with a nucleic acid, and the nucleic acid is associated with an analyte. In some instances, a complex can comprise two probes each comprising a tag, wherein the first tag associates (e.g., by ligation) with a first end of a nucleic acid and the second tag associates with a second end of a nucleic acid, and the nucleic acid is associated with an analyte. For example, a first probe comprising a first tag and a second probe comprising a second tag can be in spatial proximity if the first tag is allowed to ligate to a first end of a nucleic acid associated with an analyte, and the second tag is allowed to ligate to a second end of the nucleic acid associated with the analyte. For example, a first probe comprising a first tag and a second probe comprising a second tag can be in spatial proximity if the first tag can associate with a first end or portion of a nucleic acid associated with an analyte, and the second tag can associate with a second end or portion of the nucleic acid associated with the analyte. A person having skill in the art will appreciate that a probe can form a complex with an analyte using a variety of mechanisms, including but not limited to covalent binding, non-covalent binding (e.g., electrostatic interactions, hydrogen bonding, Van der Waals forces, or hydrophobic interactions), or a combination thereof. In some embodiments, a probe can form a complex with an analyte by directly associating with the analyte. For example, a probe comprising an antibody can form a complex with an analyte by non-covalently binding the analyte at a methylation site. In some embodiments, a probe can form a complex with an analyte by indirectly associating with the analyte. For example, a probe comprising an antibody comprising a tag can form a complex with an analyte through ligation of the tag with an end of a nucleic acid associated with the analyte. In other embodiments, a complex can comprise one or more probes. In some cases, a probe can comprise one or more tags.

### Substrate

In some embodiments, the compositions disclosed herein can comprise a substrate. In some embodiments, the compositions disclosed herein can comprise a first probe, and the first probe is associated with a substrate. In some embodiments, the compositions disclosed herein can comprise a second probe, and the second probe is associated with a substrate. In some embodiments, the compositions disclosed herein can comprise a first probe and a second probe, and the first probe and the second probe are associated with a substrate. In some embodiments, a first probe and a second probe can be associated with the same or a different substrate. In some embodiments, a substrate can be a solid substrate or a semi-solid substrate (e.g., a gel or a Sepharose bead). In some embodiments, a substrate can be a planar. In some embodiments, a planar substrate can be square. In some embodiments, a planar substrate can be rectangular. In some embodiments, the planar substrate can be asymmetrical. In some embodiments, a solid substrate can be an array. For example, a planar substrate can be in the form of a rectangular array. In some embodiments, a substrate can be spherical or generally spherical. For example, a spherical substrate can be a bead. In some embodiments, a bead can be silica bead. In another example, a spherical substrate can be a polyethylene-glycol (PEG) hydrogel bead. In yet another example, a spherical substrate can be a Sepharose bead. In some embodiments, the spherical substrate can be at least 50 nanometers, at least 100 nanometers, at least 150 nanometers, at least 200 nanometers, at least 250 nanometers, at least 300 nanometers, at least 350 nanometers, at least 400 nanometers, at least 450 nanometers, at least 475 nanometers, at least 500 nanometers, at least 550 nanometers, at least 600 nanometers, at least 650 nanometers, at least 700 nanometers, at least 750 nanometers, at least 800 nanometers, at least 850 nanometers, at least 900 nanometers, at least 950 nanometers, at least 1000 nanometers, at least 1050 nanometers, at least 1100 nanometers, at least 1150 nanometers, at least 1200 nanometers, at least 1250 nanometers, at least 1300 nanometers, at least 1350 nanometers, at least 1400 nanometers, at least 1450 nanometers, at least 1500 nanometers, at least 1550 nanometers, at least 1600 nanometers, at least 1650 nanometers, at least 1700 nanometers, at least 1750 nanometers, at least 1800 nanometers, at least 1850 nanometers, at least 1900 nanometers, at least 1950 nanometers, at least 2000 nanometers, at least 2500 nanometers, at least 3000 nanometers, at least 3500 nanometers, at least 4000 nanometers, at least 4500 nanometers, or at least 5000 nanometers nanometers in diameter. In some embodiments, the spherical substrate can be about 2800 nanometers in diameter. In some embodiments, the substrate can comprise a plurality of spherical substrates of at least two different diameters. A person of ordinary skill in the art will appreciate that the substrate can be fabricated using a variety of materials. In some embodiments, a substrate can be hydrophilic. In some embodiments, a substrate can be hydrophobic. In some embodiments, a substrate can be magnetic. In some instances, magnetic substrates can be useful for isolating or separating a substrate from a mixture. In one embodiment, a magnet can be used to isolate the magnetic substrate after contacting the substrate with a sample. For example, an analyte can be separated from a sample by (a) contacting the sample with a spherical magnetic substrate comprising two or more probes capable of binding to the analyte, (b) allowing the probes to bind to the analyte, and (c) exposing the sample to a magnetic field, wherein the magnetic field separates the spherical magnetic substrate comprising the two or more probes bound to the analyte from the sample. In some embodiments, the substrate can be non-magnetic. Non-limiting examples of materials that can be used to fabricate the substrate include polymers, silica, zirconium, gels, agarose, magnetite, maghemitite, FePt, SrFe, iron, cobalt, nickel, chromium dioxide, ferrites, or a combination thereof. In some embodiments, a solid support can comprise a plurality of probes. In some embodiments, a solid support can comprise at least about 1, 2, 3, 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more probes. In some embodiments, a probe can be coupled to a solid support via a linker. In some embodiments, a solid support can comprise at least about 1, 2, 3, 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more target analyte (analyte).

### Probe Coupled to a Substrate

A probe or an analyte can be coupled to a solid support. For example, a probe can be immobilized on a solid substrate. A probe can be coupled to the solid support through covalent or non-covalent interactions. For example, a probe can be coupled to the solid support non-covalently through hydrophobic bonding, hydrogen bonding, Van der Waals interactions, ionic bonding, etc. In some instances, a probe is coupled reversibly. In some instances, a probe is coupled irreversibly.

A probe can be coupled a solid support through a functional group (e.g., a reactive group). A probe can comprise any suitable functional group for coupling to a solid support. For example, a surface of a solid support can be coated with a functional group and a probe can be attached to the solid support through the functional group. For example, a solid support can be coated with a first functional group and a probe comprising a second functional group can be attached to the solid support by binding or reacting the first and second functional groups. For example, a surface of a solid support can be coated with streptavidin and a biotinylated a probe can be attached thereto.

A probe or functional group for attachment of a probe can be deposited on a solid surface (e.g., an array or bead) by any suitable technique. Examples of solid surface materials and corresponding functional groups include gold, silver, copper, cadmium, zinc, palladium, platinum, mercury, lead, iron, chromium, manganese, tungsten, and any alloys thereof. Exemplary functional groups of solid surfaces include sulfur-containing functional groups such as thiols, sulfides, disulfides (e.g., —SR or —SSR where R is H, alkyl, or aryl), and the like; doped or undoped silicon with silanes and chlorosilanes (e.g., —SiR2Cl where R is H, alkyl, or aryl); metal oxides (e.g., silica, alumina, quartz, glass, and the like) with carboxylic acids; platinum and palladium with nitrites and isonitriles; copper with hydroxamic acids; benzophenones; acid chlorides; anhydrides; epoxides; sulfonyl groups; phosphoryl groups; hydroxyl groups; phosphonates; phosphonic acids; amino acid groups; amides; and the like (See, e.g., U.S. Pat. No. 6,413,587). A probe can optionally be coupled to a solid support through one or more bifunctional linkers (e.g., the linkers comprising one functional group capable of forming a linkage with a solid substrate and another functional group capable of forming a linkage with another linker molecule or probe). Depending on the particular application, linkers may be long or short, flexible or rigid, charged or uncharged, and/or hydrophobic or hydrophilic.

Some compositions disclosed herein can comprise a substrate that can be contacted (e.g., coated) with at least one of a polymer or a first binding partner which has an affinity for a second binding partner. A substrate can be coated for a variety of reasons, for example, to alter the hydrophilic properties of the substrate (e.g., surface wetting), to enhance or prevent binding to a ligand or binding partner, or to shield the negative space on a substrate from subsequent coatings/treatments (e.g., for micropatterning). In some embodiments, a substrate can be completely coated. In some embodiments, a substrate can be partially coated. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the substrate is coated. In some embodiments, a substrate can be coated with one or more chemical compounds (e.g., an iodoacetyl functional group). In some embodiments, at least a portion of the substrate is coated with a polymer. Non limiting examples of polymers that can be used to coat a substrate include polyethylene glycol, polymethacrylate, polymethylmethacrylate, polyethylenimine, polyvinyl alcohol, polyvinyl acetate, polystyrene, polyglutaraldehyde, polyacrylamide, agarose, chitosan, alginate, or a combination thereof. In some embodiments comprising a substrate contacted with a polymer or a first binding partner, the probe can be conjugated to a substrate chemically or enzymatically. For example, a probe comprising an antibody can be chemically conjugated to a substrate which has been at least 60% coated with polyethylene glycol. In some embodiments, at least a portion of a substrate can be coated with a first binding partner which has an affinity for a second binding partner. Non limiting examples of the first binding partner or second binding partner include antibody, immunoglobulin-binding protein, Protein A, Protein G, Protein A/G, calmodulin, glutathione, glutathione S-transferase (GST), streptavidin, avidin, maltose-binding protein, a His tag, or a combination thereof. In one example, a substrate can be a spherical substrate, and the spherical substrate can be coated with Protein G. In some embodiments, the substrate can be coated to enable or promote association between the probe and the substrate. For example, a substrate can be coated with a first binding partner which has an affinity for a second binding partner, wherein the probe comprises the second binding partner, thereby enabling association between the probe and the substrate. Any of the embodiments disclosed herein can comprise a substrate which is at least partially coated with both of a polymer and a first binding partner which has an affinity for a second binding partner. A substrate can be coated using any method known in the art. In some embodiments, coating the substrate can comprise physical modification, chemical modification, photochemical modification, graft formation, plasma treatment, covalent immobilization, the wet chemical method, Staudinger ligation, alkali hydrolysis, or a combination thereof. For example, a spherical substrate can be coated with streptavidin by covalent immobilization.

In some embodiments, the compositions disclosed herein can comprise a probe (e.g., a first probe and/or a second probe), and the probe can comprise at least one binding partner. In general, a binding partner can be used to bind the probe to the substrate. In some embodiments, a probe can comprise a binding partner, and a binding partner can bind directly or indirectly to the substrate. In other embodiments, a probe can comprise a second binding partner, and a second binding partner binds to a first binding partner, wherein the first binding partner can be coated on the substrate. In some embodiments, a probe can comprise at least one of a binding partner of a polymer coated on a substrate or a second binding partner. Non limiting examples of a binding partner, a first binding partner or a second binding partner include antibody, immunoglobulin-binding protein, Protein A, Protein G, Protein A/G, calmodulin, glutathione, glutathione S-transferase (GST), streptavidin, avidin, maltose-binding protein, a His tag, or a combination thereof. In one example, the probe can comprise a polymer, and the polymer can be covalently immobilized directly onto the substrate. In another example, a substrate can be coated with the first binding partner GST, and the probe can comprise the second binding partner glutathione.

In some embodiments, a tag disclosed herein can be an affinity tag. Examples of such affinity tags include, but are not limited to, Glutathione-S-transferase (GST), Maltose binding protein (MBP), Green Fluorescent Protein (GFP), AviTag (a peptide allowing biotinylation by the enzyme BirA and so the protein can be isolated by streptavidin), Calmodulin-tag (a peptide bound by the protein calmodulin), polyglutamate tag (a peptide binding efficiently to anion-exchange resin such as Mono-Q), FLAG-tag (a peptide recognized by an antibody), HA-tag (a peptide recognized by an antibody), His tag (generally 5-10 histidines which are bound by a nickel or cobalt chelate), Myc-tag (a short peptide recognized by an antibody, S-tag, SBP-tag (a peptide which binds to streptavidin), Softag 1, Strep-tag (a peptide which binds to streptavidin or the modified streptavidin called streptactin), TC tag (a tetracysteine tag that is recognized by FlAsH and ReAsH biarsenical compounds), V5 tag, Xpress tag, Isopeptag (a peptide which binds covalently to pilin-C protein), SpyTag (a peptide which binds covalently to SpyCatcher protein) or a combination thereof. In some instances, for example, a probe, polynucleotides, binding moiety, first end or second end can comprise a fusion tag. For example, a probe, polynucleotides, binding moiety, first end or second end can comprise a GST-tag, His-tag, FLAG-tag, T7 tag, S tag, PKA tag, HA tag, c-Myc tag, Trx tag, Hsv tag, CBD tag, Dsb tag, pelB/ompT, KSI, MBP tag, VSV-G tag, 3-Gal tag, GFP tag, or a combination thereof, or other similar tags.

### Methods

In one aspect, provided herein are methods comprising: contacting a sample comprising a nucleic acid associated with an analyte with a first probe wherein the first probe comprises a first tag comprising a polynucleotide with a region for attaching to a first end of the nucleic acid, wherein the first tag is double stranded, and a second probe, wherein the second probe comprises a second tag comprising a polynucleotide with a region for attaching to a second end of the nucleic acid, and wherein the first probe has an affinity to a first binding site on the analyte and the second probe has an affinity to a second binding site of an analyte, and wherein the first probe and the second probe are in spatial proximity to form a complex with the analyte. In some embodiments, the first probe can be associated with a solid substrate. In other embodiments, the first probe and the second probe can be associated with the solid substrate.

In another aspect, provided herein are methods comprising: contacting a sample comprising a nucleic acid associated with an analyte with a first probe coupled to a solid substrate, wherein the first probe comprises a first tag with a region for attaching to a first end of the nucleic acid and a second probe coupled to the solid substrate, wherein the second probe comprises a second tag comprising a polynucleotide comprising a region for attaching to a second end of the nucleic acid, and wherein the first probe has an affinity to a first binding site on the analyte and the second probe has an affinity to a second binding site of the analyte, and wherein the first probe and the second probe are in spatial proximity to form a complex with the analyte.

In another aspect, provided herein are methods comprising: extracting an analyte from a sample comprising a nucleic acid associated with the analyte by contacting the sample with an extraction complex comprising an extraction moiety and an oligonucleotide, wherein the extraction complex binds to the nucleic acid; and contacting the extracted analyte with a first probe that has an affinity to a first binding site on the analyte, and a second probe that has an affinity to a second binding site on the analyte, wherein the first probe comprises a first tag with a region for attaching to a first end of the nucleic acid, and the second probe comprises a second tag comprising a polynucleotide comprising a region for attaching to a second end of the nucleic acid, and wherein the first probe and the second probe are in spatial proximity to form a complex with the analyte.

In yet another aspect, provided herein are methods comprising: coupling a solid substrate to a first probe and a second probe, wherein the first probe comprises a first tag comprising a polynucleotide and the second probe comprises a second tag comprising a polynucleotide, wherein the first probe has an affinity to a first binding site on an analyte, and the second probe binds to a second binding site on the analyte, wherein the first tag comprises a region for attaching to a first end of a nucleic acid associated with the analyte, and the second tag comprises a region for attaching to a second end of the nucleic acid associated with the analyte.

In another aspect, one or more target analytes can be comprises on a solid support. A tagged probe as described herein can be introduced to the solid support. A wash can be performed to remove unbound tagged probes. A tagged sequence of tagged probes bound to the target analyte can be amplified and/or sequenced to identify a target analyte based on the tagged sequence.

In other embodiments, one or more target analytes can be comprises on a solid support. A probe comprising an oligo as described herein can be introduced to the solid support. A wash can be performed to remove unbound probes. An oligo comprising a barcode sequence can be introduced to the solid substrate. In some cases, the barcode sequence can be unique to a specific substrate, for example, unique to each well in a multiwell substrate. In a reaction, the oligo comprising a barcode sequence can be hybridize or associate with the oligo of the probe. In an amplification and/or sequencing reaction barcode sequences that hybridized or associated with probes bound to a target analyte can be amplified and/or sequenced to identify a target analyte.

### Sample

In some embodiments, the methods disclosed herein can comprise a sample. For any of the methods disclosed herein, a sample can be obtained invasively (e.g., tissue biopsy) or non-invasively (e.g., venipuncture). In some embodiments, a sample can be a solid sample or a liquid sample. In some embodiments, a sample can be a biological sample or a non-biological sample. In some embodiments, a sample can be an in-vitro sample or an ex-vivo sample. Non-limiting examples of a sample include amniotic fluid, bile, breast milk, cells, cerebrospinal fluid, chromatin DNA, ejaculate, nucleic acids, RNA, saliva, semen, blood, serum, synovial fluid, tears, tissue, urine, whole blood or plasma, and/or any combination and/or any fraction thereof. In one example, the sample can be a plasma sample, and the plasma sample can comprise DNA. In another example, the sample can be a cell sample, and the cell sample can comprise chromatin.

In some embodiments, a sample can be a mammalian sample. In some embodiments, a sample can be a human sample. In some embodiments, a sample can be a non-human sample. Non-limiting examples of a non-human sample include a cat sample, a dog sample, a goat sample, a guinea pig sample, a hamster sample, a mouse sample, a pig sample, a non-human primate sample (e.g., a gorilla sample, an ape sample, an orangutan sample, a lemur sample, or a baboon sample), a rat sample, a sheep sample, a cow sample, or a zebrafish sample.

### Cross-Linked Nucleic Acid

In some embodiments, a nucleic acid can be cross-linked. Cross-linking of the nucleic acid can be performed in order to preserve, detect, and/or quantify an interaction between an analyte and/or a nucleic acid. In some embodiments, cross-linking can occur between the nucleic acid and the analyte. In some embodiments, the cross-linking can occur between two different positions in the nucleic acid. For example, any of the methods disclosed herein can further comprise cross-linking a nucleic acid to an analyte in order to stabilize the interaction between the nucleic acid and the analyte. In some embodiments, the cross-linking can be photochemical cross-linking. Photochemical cross-linking can comprise the introduction of photoactivatable compounds into the nucleic acid, the analyte, or a combination thereof. In some embodiments, the cross-linking can be ultraviolet cross-linking. Ultraviolet cross linking can comprise the irradiation of analyte-nucleic acid complexes with ultraviolet light, thereby causing covalent bonds to form between the nucleic acid and analytes that are in close contact with the nucleic acid. In some embodiments, the methods provided herein comprise cross-linking the nucleic acid to the analyte using a cross-linking agent. In some embodiments, the cross-linking agent can be endogenous. In some embodiments, the cross-linking agent can be exogenous. Non-limiting examples of a cross-linking agent include aldehyde, formaldehyde, paraformaldehyde, malondialdehyde, crotonaldehyde, an alkylating agent, cisplatin, nitrous acid, psoralen, or a combination thereof. Additional agents can be added to terminate the cross-linking reaction. In one example, glycine can be added to quench the formaldehyde and terminate the cross-linking reaction.

Chemical cross-linking can include the use of cross-linking agents. Suitable crosslinking agents include cisplatin, dimethyl adipimidate (DMA), dimethyl pimelimidate (DMP), dimethyl suberimidate (DMS), disuccinimidyl suberate (DSS), disuccinimidyl glutarate (DSG), ethylene glycol bis(succinimidylsuccinate) (EGS), Tris-succinimidyl aminotriacetate (TSAT), and formaldehyde. Additional cross-linking agents include alkylating agents (e.g., 1,3-bis(2-chloroethyl)-1-nitrosourea, nitrogen mustard), nitrous acid, malondialdehyde, psoralens, and aldehydes (e.g., acrolein, crotonaldehyde).

### Nucleic Acid Modification

In some embodiments, the methods disclosed herein can comprise modifying a nucleic acid associated with an analyte. For example, a nucleic acid can be modified to facilitate ligation with a polynucleotide. Modifying a nucleic acid can be performed by any method known in the art, and can comprise the use of an enzyme, an endonuclease, an exonuclease, a glycosylase, a kinase, a ligase, a methyltransferase, a nuclease, a phosphatase, a polymerase, a transferase, or a combination thereof. In some embodiments, the modifying comprises generating a single stranded overhang at least one end of a nucleic acid associated with the analyte. In some embodiments, the nucleic acid overhang can occur on the 3′ end of the nucleic acid. In some embodiments, the nucleic acid overhang can occur on the 5′ end of the nucleic acid. The nucleic acid overhang can comprise any number of nucleotides. For example, the nucleic acid overhang can be 1, 2, 3, 4, 5, 6, 7, 8, 9, 10, 11, 12, 13, 14, 15 or more nucleotides. In some embodiments, the modifying comprises generating a blunt end at least one end of the nucleic acid associated with the analyte. Non-limiting examples of enzymes or endonucleases that can be used to modify the nucleic acid include an AatII endonuclease, an Acc65I endonuclease, an AccI endonuclease, an AclI endonuclease, an AatII endonuclease, an Acc65I endonuclease, an AccI endonuclease, an AclI endonuclease, an AfeI endonuclease, an AflII endonuclease, an AgeI endonuclease, an ApaI endonuclease, an ApaLI endonuclease, an ApoI endonuclease, an AscI endonuclease, an AseI endonuclease, an AsiSI endonuclease, an AvrII endonuclease, a BamHI endonuclease, a BclI endonuclease, a BglII endonuclease, a Bme1580I endonuclease, a BmtI endonuclease, a BsaI endonuclease, a BsaHI endonuclease, a BsiEI endonuclease, a BsiWI endonuclease, a BspEI endonuclease, a BspHI endonuclease, a BsrGI endonuclease, a BssHII endonuclease, a BstBI endonuclease, a BstZ17I endonuclease, a BtgI endonuclease, a ClaI endonuclease, DraI endonuclease, an EaeI endonuclease, an EagI endonuclease, an EcoRI endonuclease, an EcoRV endonuclease, an FseI endonuclease, an FspI endonuclease, an HaeII endonuclease, an HincII endonuclease, an HindIII endonuclease, an HpaI endonuclease, a KasI endonuclease, a KpnI endonuclease, an MfeI endonuclease, an MluI endonuclease, an MscI endonuclease, an MspA1I endonuclease, an MfeI endonuclease, an MluI endonuclease, an MscI endonuclease, an MspA1I endonuclease, an NaeI endonuclease, an NarI endonuclease, an NcoI endonuclease, an NdeI endonuclease, an NgoMIV endonuclease, an NheI endonuclease, an NotI endonuclease, an NruI endonuclease, an NsiI endonuclease, an NspI endonuclease, a Pad endonuclease, a PciI endonuclease, a PmeI endonuclease, a PmlI endonuclease, a PsiI endonuclease, a PspOMI endonuclease, a PstI endonuclease, a PvuI endonuclease, a PvuII endonuclease, an SacI endonuclease, an SacII endonuclease, an SalI endonuclease, an SbfI endonuclease, an ScaI endonuclease, an SfcI endonuclease, an SfoI endonuclease, an SgrAI endonuclease, an SmaI endonuclease, an SmlI endonuclease, an SnaBI endonuclease, an SpeI endonuclease, an SphI endonuclease, an SspI endonuclease, an StuI endonuclease, an SwaI endonuclease, an XbaI endonuclease, an XhoI endonuclease, and an XmaI endonuclease. In a particular example, a BsaI enzyme can be used to modify at least one end of the nucleic acid. More than one enzyme can be used to modify a nucleic acid. In some embodiments, 2 enzymes, 3 enzymes, 4 enzymes, or 5 or more enzymes can be used to modify the nucleic acid. For example, a BsaI enzyme can be used to modify the first end of the nucleic acid, while a NotI enzyme is used to modify the second end of the nucleic acid. In some embodiments, more than 1 enzyme can be used to modify the same end of the nucleic acid. For example, digestion with a first restriction enzyme can generate a recleavable blunt end that can be digested with a second restriction enzyme.

Modifying the nucleic can result in a nucleic acid having a length that is the same (e.g., same number of nucleotides) as the nucleic acid before modifying. In some embodiments, modifying the nucleic acid can alter the length of the nucleic acid. For example, the modified nucleic acid can be larger (e.g., more nucleotides) than the nucleic acid before modifying. In some embodiments, modifying the nucleic acid can result in a nucleic acid with at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 15 nucleotides, at least 20 nucleotides, at least 25 nucleotides, at least 50 nucleotides, at least 75 nucleotides, at least 100 nucleotides, at least 500 nucleotides, or at least 1000 nucleotides more than the nucleic acid before modifying. In another example, the modified nucleic acid can be smaller (e.g., less nucleotides) than the nucleic acid before modifying. In some embodiments, modifying the nucleic acid can result in a nucleic acid with at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 15 nucleotides, at least 20 nucleotides, at least 25 nucleotides, at least 50 nucleotides, at least 75 nucleotides, at least 100 nucleotides, at least 500 nucleotides, or at least 1000 nucleotides less than the nucleic acid before modifying.

In some instances, the nucleic acid associated with the analyte can be repaired with executing one or more of the methods disclosed herein. For example, cross-linking the nucleic acid to the analyte can cause damage to the nucleic acid. In some embodiments, the methods disclosed herein comprise modifying the nucleic acid, and modifying the nucleic acid comprises repairing the nucleic acid associated with the analyte. Repairing the nucleic acid can be performed by any method known in the art, and can comprise the use of an enzyme, an endonuclease, an exonuclease, a glycosylase, a kinase, a ligase, a methyltransferase, a nuclease, a phosphatase, a polymerase, a transferase, or a combination thereof.

### Extraction Complex

The methods disclosed herein can further comprise extracting the nucleic acid from the sample. In some embodiments, the nucleic acid associated with the analyte can be extracted by contacting the sample with an extraction complex. In some embodiments, the extraction complex can comprise an extraction moiety. In general, an extraction moiety can be anything that can be used to extract or isolate a target nucleic acid. In some embodiments, the extraction moiety can be a biotin molecule. Non limiting examples of extraction moieties include avidin, beads, biotin, carbohydrates, cofactors, enzymes, enzyme inhibitors, lectins, receptor molecules, streptavidin, and any combination thereof. In other embodiments, the extraction moiety can be a combination of high affinity binding partners, such as biotin and streptavidin. High affinity binding partners can refer to any combination of molecules wherein one molecule binds to at least one other molecule with a high affinity. Non limiting examples of high-affinity binding partners include biotin and avidin (or streptavidin), carbohydrates and lectins, effector and receptor molecules, cofactors and enzymes, and enzyme inhibitors and enzymes. In some embodiments, the extraction moiety can be magnetic. In some embodiments, the extraction moiety can be non-magnetic.

In some embodiments, the extraction complex can comprise an oligonucleotide. In general, an oligonucleotide can be used to target and/or bind the nucleic acid associated with the analyte. The oligonucleotide can bind to the nucleic acid using any method known in the art. In some examples, the oligonucleotide can bind to the nucleic acid through ligation, hybridization, or any combination thereof. In some embodiments, the extraction complex can comprise 1 oligonucleotide. In some embodiments, the extraction complex can comprise 2 oligonucleotides. In some embodiments, the extraction complex can comprise a plurality of oligonucleotides (e.g., 3 or more oligonucleotides). Each oligonucleotide can comprise a plurality of nucleotides. In some embodiments, the oligonucleotide can comprise at least 5, at least 10, at least 15, at least 20, at least 25, at least 30, at least 35, at least 40, at least 45, at least 50, at least 75, or at least 100 nucleotides.

An oligonucleotide can comprise an endonuclease recognition site to facilitate ligation with the nucleic acid associated with the analyte. In some embodiments, the endonuclease recognition site can be complementary to at least one endonuclease recognition site on the nucleic acid associated with the analyte. In some embodiments, each oligonucleotide can comprise 1 endonuclease recognition site. In some embodiments, each oligonucleotide can comprise 2 endonuclease recognition sites. In some embodiments, each oligonucleotide can comprise a plurality of endonuclease recognition sites (e.g., 3 or more endonuclease recognition sites). In some embodiments, the endonuclease recognition site can be a Type I endonuclease recognition site, a Type II endonuclease recognition site, a Type III endonuclease recognition site, a Type IV endonuclease recognition site, or a Type V endonuclease recognition site. Non-limiting examples of endonuclease recognition sites include an AatII site, an Acc65I site, an AccI site, an AclI site, an AatII site, an Acc65I site, an AccI site, an AclI site, an AfeI site, an AflII site, an AgeI site, an ApaI site, an ApaLI site, an ApoI site, an AscI site, an AseI site, an AsiSI site, an AvrII site, a BamHI site, a BclI site, a BglII site, a Bme1580I site, a BmtI site, a BsaI site, a BsaHI site, a BsiEI site, a BsiWI site, a BspEI site, a BspHI site, a BsrGI site, a BssHII site, a BstBI site, a BstZ17I site, a BtgI site, a ClaI site, a DraI site, an EaeI site, an EagI site, an EcoRI site, an EcoRV site, an FseI site, an FspI site, an HaeII site, an HincII site, a HindIII site, an HpaI site, a KasI site, a KpnI site, an MfeI site, an MluI site, an MscI site, an MspA1I site, an MfeI site, an MluI site, an MscI site, an MspA1I site, an NaeI site, a NarI site, an NcoI site, an NdeI site, an NgoMIV site, an NheI site, a NotI site, an NruI site, an NsiI site, an NspI site, a PacI site, a PciI site, a PmeI site, a PmlI site, a PsiI site, a PspOMI site, a PstI site, a PvuI site, a PvuII site, a SacI site, a SacII site, a SalI site, an SbfI site, an ScaI site, an SfcI site, an SfoI site, an SgrAI site, an SmaI site, an SmlI site, an SnaBI site, an SpeI site, an SphI site, an SspI site, an StuI site, an SwaI site, an XbaI site, an XhoI site, and an XmaI site. In a particular example, the extraction complex can comprise 2 oligonucleotides, and each oligonucleotide can comprise a BsaI endonuclease recognition site. In another example, the extraction complex can comprise 2 oligonucleotides, wherein the first oligonucleotide comprises a BsaI endonuclease recognition site and the second oligonucleotide comprises a NotI endonuclease recognition site.

In some embodiments, a nucleic acid associated with the analyte can be modified by attaching an overhang or a linker to one or both ends of the nucleic acid. Linkers or overhangs may comprise nucleic acids (e.g., RNA, DNA, and RNA-DNA hybrids), peptide nucleic acids (PNAs), that comprise purine and pyrimidine bases, or other natural, chemically or biochemically modified, non-natural, or derivatized nucleotide bases. The nucleic acids may be single-stranded or double-stranded. The linker or overhang may be a single nucleotide (e.g., deoxyadenosine, deoxycytosine, deoxyguanosine, deoxythymidine). The linker maycontain only one type of nucleotide (e.g., oligodT or oligodA). The linker or overhang may contain two or more different nucleotides. The linker or overhang may be about 5 to about 50 nucleotides, about 5 to about 40 nucleotides, about 5 to 30 nucleotides. The linker or overhang may be attached to the target nucleic acid by ligation (e.g., blunt end ligation, sticky end ligation), hybridization, or PCR. One or more linkers or overhangs may be attached to the target nucleic acid. The linkers or overhangs may be attached to one or both ends of the target nucleic acid. In one example, the linkers or overhangs are non-complementary.

In some embodiments, an extraction complex can comprise a polynucleotide linker. In general, a polynucleotide linker can be used to link an extraction moiety to an oligonucleotide. A polynucleotide linker can comprise a plurality of nucleotides. In some embodiments, the polynucleotide linker can comprise at least 1, at least 5, at least 10, at least 20, at least 30, at least 40, at least 50, at least 60, at least 70, at least 80, at least 90, at least 100, at least 150, at least 200, at least 250, at least 500, or at least 1000 nucleotides. In some embodiments, a polynucleotide linker may not be cleavable. In some embodiments, a polynucleotide linker can be cleavable. For example, a polynucleotide linker can be a cleavable thiocarbonate linker.

Extracting a nucleic acid can comprise a plurality of steps including, but not limited to, contacting the sample with an extraction complex, cell lysis, detergent washes, degradation of undesired proteins (e.g., protease treatment), treatment with chelating agents, and/or purification. In some embodiments, a nucleic acid can be extracted while associated with an analyte. In some embodiments, a nucleic acid can be extracted after dissociation from an analyte. Extracting a nucleic acid can comprise any method known in the art (e.g., centrifugation, chromatin immunoprecipitation (ChIP), chromatography, crystallization, decantation, ethanol precipitation, evaporation, filtration, fractional distillation, immunoprecipitation, magnetic separation, phenol chloroform extraction, precipitation funnel separation, simple distillation, sublimation. Extracting a nucleic acid can comprise a combination of the methods disclosed herein. For example, extracting a nucleic acid associated with an analyte can comprise a combination of contacting a sample with an extraction complex comprising an extraction moiety (e.g., a magnetic bead) and an oligonucleotide, wherein the oligonucleotide is allowed to bind and or couple to the nucleic acid, followed by magnetic separation to separate the nucleic acid associated with the analyte from the sample.

Dissociating Nucleic Acid from Extraction Complex

Any of the methods disclosed herein can comprise dissociating a nucleic acid associated with an analyte from the extraction complex. In some embodiments, dissociating a nucleic acid from an extraction complex can comprise enzymatic digestion. In some embodiments, dissociating a nucleic acid from an extraction complex can comprise using a restriction enzyme, wherein the restriction enzyme specifically targets an endonuclease recognition site located on the nucleic acid, oligonucleotide and/or polynucleotide linker. For example, a nucleic acid can be dissociated from an analyte by enzymatically digesting the extraction complex with a Bsa1 restriction enzyme, wherein the extraction complex comprises an oligonucleotide comprising a BsaI endonuclease recognition site. The nucleic acid can be dissociated from an extraction complex using any method known in the art. In some embodiments, dissociating a nucleic acid from the extraction complex can comprise shearing, sonication, enzymatic digestion, DNA transposition (e.g., using a transposase), or any combination thereof.

The methods disclosed herein can further comprise dissociating a nucleic acid associated with an analyte from the analyte. A nucleic acid can be dissociated from an analyte using any method known in the art. In some embodiments, a nucleic acid can be dissociated from an analyte using a protease. For example, the nucleic acid can be dissociated from the analyte by contacting a sample with Proteinase K, a broad spectrum serine protease that can be used to digest proteins. In some embodiments, proteases can be used in combination with denaturing agents. Non-limiting examples of denaturing agents include chelating agents, chymotrypsin, EDTA, sodium dodecyl sulfate (SDS), trypsin, urea or any combination thereof. In a particular example, the nucleic acid can be dissociated from the analyte (e.g., a histone) by contacting the sample comprising the nucleic acid with a co-formulation of Proteinase K and SDS.

Attaching a First Tag to a First End or Portion of a Nucleic Acid Associated with an Analyte

Some methods disclosed herein generally describe a method of contacting a sample comprising an analyte associating nucleic acid with a composition comprising a first probe and a second probe, wherein the first probe comprises a first tag and the second probe comprises a second tag. In some embodiments, the methods disclosed herein can further comprise attaching the first tag to a first end or portion of a nucleic acid associated with an analyte and a second tag to a second end or portion of a nucleic acid associated with an analyte. In some embodiments, the attaching can comprise ligation or hybridization, wherein complementary ends of a nucleic acid and a tag are annealed. In some embodiments, the attaching can comprise proximity ligation. In general, proximity ligation can refer to a technique where nucleic acids are in close enough proximity to interact stochastically, chemically or enzymatically. For example, when a first probe comprising a first tag is bound to a first binding region on an analyte comprising a nucleic acid associated with the analyte, the first tag can be in close proximity to the first end of the nucleic acid to interact (e.g., ligate with the first end of the nucleic acid). In another example, when a second probe comprising a second tag bound to a second binding region on an analyte comprising a nucleic acid associated with the analyte, the second tag can be in close proximity to the second end of the nucleic acid to interact (e.g., ligate with the second end of the nucleic acid). In some embodiments, the attaching can comprise a Class 6 enzyme or any one or more enzyme disclosed herein. In some embodiments, the attaching can comprise a ligase, a synthetase, a lyase, or any combination thereof. Non-limiting examples of ligases include DNA ligase I, DNA ligase III, DNA ligase IV, blunt/TA ligase, T3 ligase, T4 ligase, T7 ligase, Taq ligase, electroligase, E. coli ligase, 9º N ligase, SplintR ligase, tRNA ligase, Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9º N DNA Ligase, Tsp DNA ligase, novel ligases discovered by bioprospecting, and any combination thereof. For example, attaching a first tag to a first end of a nucleic acid can be performed using a T4 ligase. In another example, attaching a first tag to a first end of a nucleic acid and a second tag to a second end of the nucleic acid can be performed using the same ligase (e.g., a T4 ligase). In yet another example, attaching a first tag to a first end of a nucleic acid can be performed using a first ligase, and attaching a second tag to a second end of the nucleic acid can be performed using a second ligase. In some embodiments, the first ligase and the second ligase can be different or the same.

In some embodiments the attached first tag to a first end or portion of a nucleic acid and or an attached second tag to a second end or portion of a nucleic acid can be released (e.g. released complex) from a support (e.g. beads). In some embodiments, the released complex can comprise a first barcode, a nucleic acid associated with an analyte and a second barcode. In some embodiments, the released complex can comprise a first barcode, a nucleic acid that was associated with an analyte and a second barcode but not an analyte. In some embodiments, the released complex can comprise a first barcode. In some embodiments, the released complex can comprise a nucleic acid that was associated with an analyte. In some embodiments, the released complex can be released by any method disclosed herein for example by heating, desalting column, digestion, proteinase K digestion or any combination of techniques disclosed herein. The methods disclosed herein can further comprise dissociating a nucleic acid associated with an analyte from the analyte. A nucleic acid can be dissociated from an analyte using any method known in the art. In some embodiments, a nucleic acid can be dissociated from an analyte using a protease. For example, the nucleic acid can be dissociated from the analyte by contacting a sample with Proteinase K, a broad spectrum serine protease that can be used to digest proteins. In some embodiments, proteases can be used in combination with denaturing agents. Non-limiting examples of denaturing agents include chelating agents, chymotrypsin, EDTA, sodium dodecyl sulfate (SDS), trypsin, urea or any combination thereof. In a particular example, the nucleic acid can be dissociated from the analyte (e.g., a histone) by contacting the sample comprising the nucleic acid with a co-formulation of Proteinase K and SDS.

### Amplification

Any method disclosed herein can comprise analyzing a nucleic acid associated with an analyte. In some embodiments, a nucleic acid can be dissociated from an analyte. In some embodiments, a nucleic acid that has been dissociated from an analyte can be flanked on one end by a first barcode and flanked on the other end by a second barcode. In some embodiments, a nucleic acid that has been dissociated from an analyte can be flanked on one end by a first barcode and flanked on the other end by a second barcode can be analyzed via amplification and/or sequencing. In some embodiments, a nucleic acid that has been dissociated from an analyte can be flanked on one end by a first barcode can be analyzed via amplification and/or sequencing. In some embodiments, analyzing a nucleic acid can comprise amplifying the nucleic acid, sequencing the nucleic acid, detection of epigenetic markers (e.g., methylation, hydroxymethylation) or any combination thereof. In some embodiments, the methods disclosed herein can comprise bisulfite sequencing. In some embodiments, the analyzing can comprise amplifying the nucleic acid. Amplification of the nucleic acid can generally refer to a process by which one or more nucleic acids can be copied, thereby generating an amount of copies of the nucleic acid that can be multiple orders of magnitude greater than the starting number of nucleic acids. For example, amplification can be used in any of the methods disclosed herein for increasing the number of copies of the nucleic acid bound to the analyte in the sample. A person having skill in the art will appreciate that amplification of a nucleic acid can be performed by a variety of techniques. Non-limiting examples of amplification techniques include reverse transcription-PCR, real-time PCR, quantitative real-time PCR, digital PCR (dPCR), digital emulsion PCR (dePCR), clonal PCR, amplified fragment length polymorphism PCR (AFLP PCR), allele specific PCR, assembly PCR, asymmetric PCR (in which a great excess of primers for a chosen strand can be used), colony PCR, helicase-dependent amplification (HDA), Hot Start PCR, inverse PCR (IPCR), in situ PCR, long PCR (extension of DNA greater than about 5 kilobases), multiplex PCR, nested PCR (uses more than one pair of primers), single-cell PCR, touchdown PCR, loop-mediated isothermal PCR (LAMP), recombinase polymerase amplification (RPA), and nucleic acid sequence based amplification (NASBA).

Other amplification methods include LCR (ligase chain reaction) which utilizes DNA ligase, and a probe consisting of two halves of a DNA segment that is complementary to the sequence of the DNA to be amplified, enzyme QB replicase and an RNA sequence template attached to a probe complementary to the DNA to be copied which is used to make a DNA template for exponential production of complementary RNA, strand displacement amplification (SDA), multiple displacement amplification, ramification amplification, Qβ replicase amplification (QβRA), self-sustained replication (3 SR), Branch DNA Amplification, Rolling Circle Amplification, Circle to Circle Amplification, SPIA amplification, Target Amplification by Capture and Ligation (TACL) amplification, and RACE amplification. One commonly used technique for nucleic acid amplification is standard PCR. In general, standard PCR is a process of nucleic acid amplification that involves an enzymatic chain reaction for preparing exponential quantities of a specific nucleic acid sequence. Specifically, standard PCR involves cycling the temperature of the reaction to denature nucleic acids into single strands, anneal primers to regions of the nucleic acid that are complementary to the primer, and copy the denatured nucleic acid by extension or elongation from the primer using an enzyme and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed. Because PCR requires a small amount of starting nucleic acid material to initiate the chain reaction, the technique is particularly useful for assaying samples with low nucleic acid content.

In some embodiments, the analyzing can be performed at a single temperature. For example, analyzing the nucleic acid can comprise PCR, and the PCR can be performed at 72 degrees Celsius. In some embodiments, the analyzing can be performed at about 20 degrees Celsius, about 25 degrees Celsius, about 30 degrees Celsius, about 35 degrees Celsius, about 40 degrees Celsius, about 45 degrees Celsius, about 50 degrees Celsius, about 55 degrees Celsius, about 60 degrees Celsius, about 65 degrees Celsius, about 70 degrees Celsius, about 75 degrees Celsius, about 80 degrees Celsius, about 85 degrees Celsius, about 90 degrees Celsius, about 95 degrees Celsius, about 100 degrees Celsius, or greater than about 100 degrees Celsius. In some embodiments, the analyzing can be performed at multiple temperatures. For example, the analyzing can comprise performing PCR, and the PCR reaction can comprise a first step (e.g., denaturation) at a first temperature, a second step (e.g., annealing) at a second temperature, and a third step (e.g., extension or elongation) at a third temperature. A person having skill in the art will appreciate that the PCR reaction can comprise any number of steps, each step being performed at a given temperature. In some embodiments, at least two steps can be performed at the same temperature. In some embodiments, at least two steps can be performed at different temperatures. For example, the analyzing can comprise performing PCR, and the PCR reaction can comprise a denaturation step at about 95 degrees Celsius, an annealing step at about 55 degrees Celsius, and an extension step at about 75 degrees Celsius. In some embodiments, the analyzing can comprise multiple cycles of multiple temperatures. In some embodiments, the analyzing can comprise at least 5 cycles. In some embodiments, the analyzing can comprise about 10, about 15, about 20, about 25, about 30, about 35, about 40, about 45, or about 50 cycles. In some embodiments, the analyzing can comprise greater than about 50 cycles. In some embodiments, each cycle can comprise any number of steps, performed at any number of different temperatures. For example, the analyzing can comprise performing PCR, and the PCR reaction can comprise performing 25 cycles, wherein one cycle constitutes performing a denaturation step followed by an annealing step followed by an extension step. In some embodiments, the analyzing can comprise multiple cycles, each cycle can comprise multiple steps, and each step within a given cycle can occur over any amount of time. For example, the analyzing can comprise performing PCR, and the PCR reaction can comprise performing 30 cycles, wherein one cycle constitutes performing a denaturation step for 2 minutes followed by an annealing step for 1 minute followed by an extension step for 1 minute. Any step within a cycle can be performed for any amount of time. In some embodiments, a step can be performed for at most about 5 seconds. In some embodiments, a step can be performed for at least about 5 second, at least about 10 seconds, at least about 20 seconds, at least about 30 seconds, at least about 45 seconds, at least about 60 seconds, at least about 90 seconds, at least about 120 seconds, at least about 150 seconds, at least about 180 seconds, at least about 210 seconds, at least about 240 seconds, at least about 270 seconds, or at least about 300 seconds. In some embodiments, a step can be performed for greater than about 300 seconds.

In some embodiments, analyzing can require the use of a primer. A primer generally refers to a short synthetic nucleic acid molecule whose sequence matches a region flanking the target nucleic acid that should be amplified. In some embodiments, a primer can be between 10 and 50 nucleotides in length, inclusive. In some embodiments, a primer can be less than 10 nucleotides in length. In some embodiments, a primer can be greater than 50 nucleotides in length. Primers can comprise any number of adenine (A), thymine (T), guanine (G), cytosine (C), or uracil (U) nucleotides. In some embodiments, the type, number and arrangement of each of the nucleotides in the primer can affect the affinity between the primer and a primer binding site and/or the temperature at which the primer can bind to a primer binding site. For example, the guanine-cytosine (e.g., GC content) is the percentage of nitrogenous bases on a DNA molecule that are either guanine or cytosine, and can be used to predict the temperature at which the primer anneals to a nucleic acid. In some embodiments, the GC-content of the primer can be about 60%. In some embodiments, the GC-content of the primer can be between 50% and 60%, inclusive. In some embodiments, the GC content can be at least 10%, at least 20%, at least 30%, at least 40%, at least 50%, at least 60%, at least 70%, at least 80%, or at least 90%. In some embodiments, a primer can be a universal primer. A universal primer contains a unique amplification or sequencing priming region that is, for example, about 5, 7, 10, 13, 15, 17, 20, 22, or 25 nucleotides in length, and is present on each polynucleotide of a plurality of polynucleotides to be amplified. Thus, a universal primer can be used to amplify multiple polynucleotides simultaneously, in a single reaction, and/or with similar amplification efficiencies. In some embodiments, the primer can be conjugated with another molecule (e.g., a ribozyme), thereby allowing the primer to bind to a nucleic acid and self-cleave at a designated endonuclease recognition site. In some embodiments, the attached molecule can be temperature sensitive and/or pH sensitive. For example, analyzing a nucleic can comprise PCR amplification of the nucleic acid, wherein a ribozyme-conjugated primer is used to bind to the nucleic acid to allow repeated replication until the temperature is changed (e.g., increased or decreased) and the molecule is activated, thereby terminating replication.

### Sequencing

In some embodiments, the analyzing can comprise sequencing a nucleic acid. Sequencing the nucleic acid can be performed using any method known in the art. In some embodiments, sequencing can include next generation sequencing. In some embodiments, sequencing the nucleic acid can be performed using chain termination sequencing, hybridization sequencing, Illumina sequencing, ion torrent semiconductor sequencing, mass spectrophotometry sequencing, massively parallel signature sequencing (MPSS), Maxam-Gilbert sequencing, nanopore sequencing, polony sequencing, pyrosequencing, shotgun sequencing, single molecule real time (SMRT) sequencing, SOLiD sequencing, or any combination thereof. In some embodiments, the analyzing can comprise sequencing, and the sequencing can be initiated from the first end of the nucleic acid comprising the first tag. In some embodiments, the analyzing can comprise sequencing, and the sequencing can be initiated from the second end of the nucleic acid comprising the second tag.

The number or the average number of times that a particular nucleotide within the nucleic acid is read during the sequencing process (e.g., the sequencing depth) can be multiple times larger than the length of the nucleic acid being sequenced. In some instances, when the sequencing depth is sufficiently larger (e.g., by at least a factor of 5) than the length of the nucleic acid, the sequencing can be referred to as ‘deep sequencing’. In any of the embodiments disclosed herein, analyzing the nucleic acid can comprise deep sequencing. For example, a nucleic acid can be sequenced such that the sequencing depth is about 20 times greater than the length of the nucleic acid. In some instances, when the sequencing depth is at least about 100 times greater than the length of the nucleic acid, the sequencing can be referred to as ‘ultra-deep sequencing’. In any of the embodiments disclosed herein, analyzing the nucleic acid can comprise ultra-deep sequencing. In some embodiments, the sequencing depth can be one average at least about 5 times greater, at least about 10 times greater, at least about 20 times greater, at least about 30 times greater, at least about 40 times greater, at least about 50 times greater, at least about 60 times greater, at least about 70 times greater, at least about 80 times greater, at least about 90 times greater, at least about 100 times greater than the length of the nucleic acid being sequenced.

### Epigenetic Markers

In some embodiments, the analyzing can comprise detecting epigenetic markers. Epigenetic markers can be any modification of a nucleic acid or an analyte associated with a nucleic acid that can affect gene transcription and/or affect protein expression. Non-limiting examples of epigenetic markers include nucleic acid methylation, nucleic acid hydroxymethylation, and histone modifications (e.g., acetylation and methylation of histone proteins). In one example, changes in the pattern of methylation or hydroxymethylation can regulate nucleic acid-analyte binding, thereby effecting changes in gene expression and causing disease (e.g. cancer). These aberrant methylation patterns can be used to detect the presence of disease in a subject. Non-limiting examples of disease that can be detected include adrenal cancer, anal cancer, B-cell lymphoma, basal cell carcinoma, bile duct cancer, bladder cancer, blood cancer, bone cancer, a brain tumor, breast cancer, cancer of the cardiovascular system, cervical cancer, colon cancer, colorectal cancer, diffuse large B-cell lymphoma, cancer of the endocrine system, esophageal cancer, eye cancer, follicular lymphoma, gallbladder cancer, a gastrointestinal tumor, kidney cancer, hematopoietic malignancy, laryngeal cancer, leukemia, liver cancer, lung cancer, lymphoma, mantle cell lymphoma, melanoma, mesothelioma, cancer of the muscular system, Myelodysplastic Syndrome (MDS), myeloma, cancer of the nasal cavity, cancer of the nervous system, cancer of the lymphatic system, lymphoplasmacytic lymphoma, oral cancer, osteosarcoma, ovarian cancer, pancreatic cancer, penile cancer, pituitary tumors, prostate cancer, rectal cancer, renal pelvis cancer, cancer of the reproductive system, cancer of the respiratory system, sarcoma, salivary gland cancer, skeletal system cancer, skin cancer, small intestine cancer, small lymphocytic lymphoma, stomach cancer, T-cell lymphoma, testicular cancer, throat cancer, thymus cancer, thyroid cancer, a tumor, cancer of the urinary system, uterine cancer, vaginal cancer, or vulvar cancer. Any of the cancers disclosed herein can be acute or chronic. In some embodiments, the subject may not be clinically diagnosed with cancer. In general, the methods disclosed herein can be used to identify epigenetic markers associated with nucleic acids bound to an analyte comprising two or more binding regions of interest, wherein the epigenetic markers can be associated with the nucleic acid and/or the analyte. In some embodiments, the analyzing can occur prior to attaching a first tag to a first end of the nucleic acid associated with an analyte and a second tag to a second end of the nucleic acid associated with the analyte. In some embodiments, the analyzing can occur after attaching a first tag to a first end of a nucleic acid associated with an analyte and a second tag to a second end of the nucleic acid associated with the analyte. In some embodiments, the analyzing can occur after attaching either a first tag to a first end of a nucleic acid associated with an analyte or a second tag to a second end of the nucleic acid associated with the analyte.

Epigenetic modifications, such as the chemical modification of nucleic acids (e.g., DNA methylation), the modification of an analyte associated with a nucleic acid (e.g., histones), or a change in the interaction between an analyte and a nucleic acid can affect the transcriptional efficiency of a given gene. Identification of correlations between the presence or absence of one or more modification with a pathological state can provide new methods for detecting, preventing, and/or prognosticating diseases in patients. In some aspects, the methods disclosed herein comprise calculating a first value of at least one parameter. In some embodiments, the at least one parameter can correspond to a transcriptional efficiency of at least a portion of the nucleic acid associated with the analyte. Transcriptional efficiency can generally refer to the rate at which genomic material (e.g., DNA) is transcribed into protein-encoding RNA. In some aspects, transcriptional efficiency can generally refer to an amount of protein-encoding RNA derived from genomic material. In some embodiments, transcriptional efficiency can be correlated to a presence of at least one of the first binding site or the second binding site on the analyte. In some embodiments, translational efficiency can be correlated to an absence of at least one of the first binding site or the second binding site on the analyte. In one example, a parameter corresponding to transcriptional efficiency can be measured by analyzing the nucleic acid associated with the analyte (e.g., performing PCR), and determining the number of amplicons that are capable of being produced, wherein the number of amplicons is an indirect measure of the transcriptional efficiency. In some embodiments, the at least one parameter can correspond to a translational efficiency of at least a portion of the nucleic acid associated with the analyte. Translational efficiency can generally refer to the rate at which genomic material (e.g., DNA) is ultimately translated into proteins, or the rate at which any intermediate step in the process occurs. In some aspects, translational efficiency can generally refer to an amount of protein derived from genomic material or RNA. In some embodiments, translational efficiency can be correlated to a presence of at least one of the first binding site or the second binding site on the analyte. In some embodiments, translational efficiency can be correlated to an absence of at least one of the first binding site or the second binding site on the analyte. For example, a method described herein can be used to correlate the presence of a combination of histone modifications with a decrease is protein production. In another example, a method described herein can be used to develop a database of modifications (e.g., post translational modification, epigenetic modification, histone modifications) correlated with the increase or decrease of a protein. In yet another example, a method described herein can be used to develop a database of modifications (e.g., post translational modification, epigenetic modification, histone modifications) correlated with the presence or absence of a disease.

In some embodiments, the methods described herein can be completely or partial performed in the liquid phase or solid phase.

### Kits

Also provided are kits that find use in practicing the subject methods, as mentioned above. In some aspects, this disclosure provides kits comprising a targeting complex. In some embodiments, a kit can comprise a first probe and a second probe. In some embodiments, a kit can comprise a substrate.

A kit can include one or more reagents for performing amplification, including suitable primers, enzymes, nucleobases, and other reagents such as PCR amplification reagents (e.g., nucleotides, buffers, cations, etc.), and the like. Additional reagents that are required or desired in the protocol to be practiced with the kit components may be present. Such additional reagents include, but are not limited to, one or more of the following an enzyme or combination of enzymes such as a polymerase, reverse transcriptase, nickase, restriction endonuclease, uracil-DNA glycosylase enzyme, enzyme that methylates or demethylates DNA, endonuclease, ligase, etc. A kit can include one or more reagents for performing sequencing.

The kit components may be present in separate containers, or one or more of the components may be present in the same container, where the containers may be storage containers and/or containers that are employed during the assay for which the kit is designed.

In addition to the above components, the subject kits may further include instructions for practicing the subject methods. These instructions may be present in the subject kits in a variety of forms, such as printed information on a suitable medium or substrate (e.g., a piece or pieces of paper on which the information is printed), in the packaging of the kit, in a package insert, etc. Yet another means would be a computer readable medium (e.g., diskette, CD, etc.), on which the information has been recorded. Yet another means that may be present is a website address which may be used via the internet to access the information at a removed site.

### Sample

The sample disclosed herein can be a sample from a healthy subject or a subject with a condition or disease. For example, a sample can be a diseased tissue or cell, such as a breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer tissue or cell. The sample can be from a subject with a disease or condition such as a cancer, inflammatory disease, immune disease, autoimmune disease, cardiovascular disease, neurological disease, infectious disease, metabolic disease, or a perinatal condition. For example, the disease or condition can be a tumor, neoplasm, or cancer. The cancer can be, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, or Burkitt's lymphoma. The disease or condition can also be a premalignant condition, such as Barrett's Esophagus. The disease or condition can also be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis. The disease or condition can also be a cardiovascular disease, such as atherosclerosis, congestive heart failure, vulnerable plaque, stroke, or ischemia. The cardiovascular disease or condition can be high blood pressure, stenosis, vessel occlusion or a thrombotic event. The disease or condition can also be a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neuropsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The condition may also be fibromyalgia, chronic neuropathic pain, or peripheral neuropathic pain. The disease or condition may also be an infectious disease, such as a bacterial, viral or yeast infection. For example, the disease or condition may be Whipple's Disease, Prion Disease, cirrhosis, methicillin-resistant staphylococcus aureus, HIV, hepatitis, syphilis, meningitis, malaria, tuberculosis, or influenza. The disease or condition can also be a perinatal or pregnancy related condition (e.g. preeclampsia or preterm birth), or a metabolic disease or condition, such as a metabolic disease or condition associated with iron metabolism.

### Supports/Substrates

Generally, a substrate can be composed of any material which will permit coupling of a probe, which will not melt or otherwise substantially degrade under the conditions used to hybridize and/or denature nucleic acids. A substrate can be composed of any material which will permit coupling of a probe or other moiety at one or more discrete regions and/or discrete locations within the discrete regions. A substrate can be composed of any material which permit washing or physical or chemical manipulation without dislodging a probe or moiety from the solid support.

Substrates can be fabricated by the transfer probes onto the solid surface in an organized high-density format followed by coupling the probe thereto. The techniques for fabrication of a substrate of the invention include, but are not limited to, photolithography, ink jet and contact printing, liquid dispensing and piezoelectrics. The patterns and dimensions of arrays are to be determined by each specific application. The sizes of each target analyte spots may be easily controlled by the users.

A method of making a solid substrate can comprise contacting or coupling a probe to a first discrete location of a discrete region on a solid support. The coupling can include any of the coupling methods described herein or otherwise known in the art. In some instances, a solid support is coated with an affinity ligand as described herein and contacting or coupling a probe thereto.

A substrate can take a variety of configurations ranging from simple to complex. A support may be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. A solid support as described above can be formed of any suitable material, including metals, metal oxides, semiconductors, polymers (particularly organic polymers in any suitable form including woven, nonwoven, molded, extruded, cast, etc.), silicon, silicon oxide, and composites thereof. A number of materials (e.g., polymers) suitable for use as substrates (e.g., solid substrates) in the instant invention have been described in the art. Suitable materials for use as substrates include, but are not limited to, polycarbonate, gold, silicon, silicon oxide, silicon oxynitride, indium, tantalum oxide, niobium oxide, titanium, titanium oxide, platinum, iridium, indium tin oxide, diamond or diamond-like film, acrylic, styrene-methyl methacrylate copolymers, ethylene/acrylic acid, acrylonitrile-butadiene-styrene (ABS), AB S/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polyacrylonitrile (PAN), polyacrylate, polycarbonate, polybutylene terephthalate (PBT), poly(ethylene) (PE) (including low density, linear low density, high density, cross-linked and ultra-high molecular weight grades), poly(propylene) (PP), cis and trans isomers of poly(butadiene) (PB), cis and trans isomers of poly(isoprene), polyethylene terephthalate) (PET), polypropylene homopolymer, polypropylene copolymers, polystyrene (PS) (including general purpose and high impact grades), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinylalcohol (PVA), polyacrylamide and its homologs such as poly(N-isopropyl acrylamide), fluorinated polyacrylate (PFOA), poly(ethylene-butylene) (PEB), poly(styrene-acrylonitrile) (SAN), polytetrafluoroethylene (PTFE) and its derivatives, polyolefin plastomers, fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene (PCTFE), polyethylene-chlorotrifluoroethylene (ECTFE), styrene maleic anhydride (SMA), metal oxides, glass, silicon oxide or other inorganic or semiconductor material (e.g., silicon nitride), compound semiconductors (e.g., gallium arsenide, and indium gallium arsenide), and combinations thereof.

Examples of well-known solid supports include polypropylene, polystyrene, polyethylene, dextran, nylon, amylases, glass, natural and modified celluloses (e.g., nitrocellulose), polyacrylamides, agaroses and magnetite. In some instances, the solid support can be silica or glass because of its great chemical resistance against solvents, its mechanical stability, its low intrinsic fluorescence properties, and its flexibility of being readily functionalized. In one embodiment, the substrate is glass, particularly glass coated with nitrocellulose, more particularly a nitrocellulose-coated slide (e.g., FAST slides).

A substrate may be modified with one or more different layers of compounds or coatings that serve to modify the properties of the surface in a desirable manner. For example, a substrate may further comprise a coating material on the whole or a portion of the surface of the substrate. In some embodiments, a coating material enhances the affinity of a probe, or another moiety (e.g., a functional group) for the substrate. For example, the coating material can be nitrocellulose, silane, thiol, disulfide, or a polymer. When the material is a thiol, the substrate may comprise a gold-coated surface and/or the thiol comprises hydrophobic and hydrophilic moieties. When the coating material is a silane, the substrate comprises glass and the silane may present terminal moieties including, for example, hydroxyl, carboxyl, phosphate, glycidoxy, sulfonate, isocyanato, thiol, or amino groups. In an alternative embodiment, the coating material may be a derivatized monolayer or multilayer having covalently bonded linker moieties. For example, the monolayer coating may have thiol (e.g., a thioalkyl selected from the group consisting of a thioalkyl acid (e.g., 16-mercaptohexadecanoic acid), thioalkyl alcohol, thioalkyl amine, and halogen containing thioalkyl compound), disulfide or silane groups that produce a chemical or physicochemical bonding to the substrate. The attachment of the monolayer to the substrate may also be achieved by non-covalent interactions or by covalent reactions.

After attachment to the substrate, the coating may comprise at least one functional group. Examples of functional groups on the monolayer coating include, but are not limited to, carboxyl, isocyanate, halogen, amine or hydroxyl groups. In one embodiment, these reactive functional groups on the coating may be activated by standard chemical techniques to corresponding activated functional groups on the monolayer coating (e.g., conversion of carboxyl groups to anhydrides or acid halides, etc.). Exemplary activated functional groups of the coating on the substrate for covalent coupling to terminal amino groups include anhydrides, N-hydroxysuccinimide esters or other common activated esters or acid halides, Exemplary activated functional groups of the coating on the substrate include anhydride derivatives for coupling with a terminal hydroxyl group; hydrazine derivatives for coupling onto oxidized sugar residues of the linker compound; or maleimide derivatives for covalent attachment to thiol groups of the linker compound. To produce a derivatized coating, at least one terminal carboxyl group on the coating can be activated to an anhydride group and then reacted, for example, with a linker compound. Alternatively, the functional groups on the coating may be reacted with a linker having activated functional groups (e.g., N-hydroxysuccinimide esters, acid halides, anhydrides, and isocyanates) for covalent coupling to reactive amino groups on the coating.

A substrate can contain a linker (e.g., to indirectly couple a moiety, probe, to the substrate). In one embodiment, a linker has one terminal functional group, and a spacer region. The terminal functional groups for reacting with functional groups on an activated coating include halogen, amino, hydroxyl, or thiol groups. In some instances, a terminal functional group is selected from the group consisting of a carboxylic acid, halogen, amine, thiol, alkene, acrylate, anhydride, ester, acid halide, isocyanate, hydrazine, maleimide and hydroxyl group. The spacer region may include, but is not limited to, polyethers, polypeptides, polyamides, polyamines, polyesters, polysaccharides, polyols, multiple charged species or any other combinations thereof. Exemplary spacer regions include polymers of ethylene glycols, peptides, glycerol, ethanolamine, serine, inositol, etc. The spacer region may be hydrophilic in nature. The spacer region may be hydrophobic in nature. In some instances, the spacer has n oxyethylene groups, where n is between 2 and 25. In some instances, a region of a linker that adheres to probe or other moiety is hydrophobic or amphiphilic with straight or branched chain alkyl, alkynyl, alkenyl, aryl, arylalkyl, heteroalkyl, heteroalkynyl, heteroalkenyl, heteroaryl, or heteroarylalkyl.

In some embodiments, a support can be planar. In some instances, the support can be spherical. In some instances, the support can be a bead. In some instances, a support can be magnetic. In some instances, a magnetic solid support can comprises magnetite, maghemitite, FePt, SrFe, iron, cobalt, nickel, chromium dioxide, ferrites, or mixtures thereof. In some instances, a support can be nonmagnetic. In some embodiments, the nonmagnetic solid support can comprise a polymer, metal, glass, alloy, mineral, or mixture thereof. In some instances a nonmagnetic material can be a coating around a magnetic solid support. In some instances, a magnetic material may be distributed in the continuous phase of a magnetic material. In some embodiments, the solid support comprises magnetic and nonmagnetic materials. In some instances, a solid support can comprise a combination of a magnetic material and a nonmagnetic material. In some embodiments, the magnetic material is at least about 5, 10, 20, 30, 40, 50, 60, 70, or about 80% by weight of the total composition of the solid support. In some embodiments, the bead size can be quite large, on the order of 100-900 microns or in some cases even up to a diameter of 3 mm. In other embodiments, the bead size can be on the order of 1-150 microns. The average particle diameters of beads of the invention can be in the range of about 2 μm to several millimeters, e.g., diameters in ranges having lower limits of 2 μm, 4 μm, 6 μm, 8 μm, 10 μm, 20 μm, 30 μm, 40 μm, 50 μm, 60 μm, 70 μm, 80 μm, 90 μm, 100 μm, 150 μm, 200 μm, 300 μm, or 500 μm, and upper limits of 20 μm, 30 μm, 40 μm, 50 μm, 60 μm, 70 μm, 80 μm, 90 μm, 100 μm, 150 μm, 200 μm, 300 μm, 500 μm, 750 μm, 1 mm, 2 mm, or 3 mm.

A support or substrate can be an array. In some embodiment a solid support comprises an array. An array of the invention can comprise an ordered spatial arrangement of two or more discrete regions.

### OTHER EMBODIMENTS

The section headings used herein are for organizational purposes only and are not to be construed as limiting the subject matter described.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims. While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Some inventive embodiments herein contemplate numerical ranges. When ranges are present, the ranges include the range endpoints. Additionally, every sub range and value within the rage is present as if explicitly written out. The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed.

### EXAMPLES

### Example 1. Identifying Regions of Genomic DNA Bound to a Histone Comprising Two Residue Modifications

A biological sample (e.g., cell lysate) comprising genomic DNA bound to histones is prepared. The genomic DNA is fragmented such that the genomic DNA bound to the histones comprise two free ends. The histone-bound genomic DNA is extracted from the biological sample with an extraction complex. The extraction complex comprises an extraction moiety (e.g., biotin), oligonucleotide sequences which bind to the free ends of the genomic DNA, cleavage sites near each oligonucleotide sequence, and polynucleotide linkers coupling the oligonucleotides to the biotin. The free ends of the genomic DNA are captured by incubation with biotin-oligonucleotide complex and a DNA ligase such that the oligonucleotide sequences ligate onto the respective free ends of the genomic DNA. The biotinylated DNA-histone complexes are then selectively extracted from the sample using streptavidin-coated microbeads. Following extraction, the oligonucleotide-labeled DNA-histone complexes are released from the extraction complexes by enzyme digestions at the cleavage sites. Chromatin immunoprecipitation (ChIP) is then used to isolate the oligonucleotide-labeled DNA-histone complexes.

Genomic DNA bound to isolated histones with two residue modifications is labeled and purified away from the histones. A solid substrate (e.g., Protein G-coated bead) comprising a first probe (e.g., antibody) with an affinity to a first histone residue modification and a second probe (e.g., antibody) with an affinity to a second histone residue modification is prepared. The first and second probes are further modified to comprise unique first and second tags, respectively. The first and second tags comprise unique polynucleotides (e.g., nucleotide barcodes) with regions for attaching to the first and second ends of the oligonucleotide-labeled DNA, respectively, as well as cleavage sites near each polynucleotide sequence. The isolated oligonucleotide-labeled DNA-histone complexes are incubated with the Protein G beads comprising antibodies specific to two residue modifications of interest. Histones comprising both residues are captured by the antibodies and proximity ligation is used to ligate the oligonucleotide-labeled DNA ends to the nucleotide barcodes on the tags. The tagged DNA (e.g., barcode-genomic DNA-barcode) is then digested at the cleavage sites to release it from the bead. The tagged DNA is then released from the histone by incubation with Proteinase K and isolated on-column. The isolated and tagged DNA sequence is then amplified (e.g., using PCR) and sequenced (e.g., using Deep-Seq) in order to identify those regions of the DNA which are bound to histones comprising the two residue modifications of interest. Such regions can then be correlated with transcription levels/expression data in order to determine how combinations of histone residue modifications can affect them.

### Example 2. Identification of Histone Modification Combinations Relevant to Disease

The approach of Example 1 is used for the identification of relevant combinations of histone modifications for disease. For a given disease of interest, Protein G beads are prepared comprising multiple combinations of antibodies specific to histone modifications known to be relevant to the disease of interest alone as well as other histone modifications which have yet to be identified as relevant to the disease on their own. Genomic DNA associated with histone modification combinations is isolated, amplified, sequenced, and analyzed for association with disease presence, absence, or severity (via informatics, in vitro testing, and/or in vivo testing). The predictive value and/or diagnostic value of the histone modification combinations for the diseases of interest are also assessed when clinical data is available or obtainable.

### Example 3. Asymptomatic Screening for Cancer in a Patient

A biological sample (e.g. blood or biopsy) comprising genomic DNA bound to histones is prepared from an asymptomatic patient as part of a cancer screen. DNA bound to histones with two or more histone modifications of interest is isolated from the biological sample as described in Example 1. The histone modifications of interest are known to be predictive of the presence, absence, and/or severity of cancer (e.g., the modifications are known to regulate known tumor suppressor and/or oncogene transcription which correlate with disease state and/or the modifications themselves correlate with disease state). The amount of isolated DNA-histone complex comprising the histone modifications of interest is quantified and used to determine if the asymptomatic patient has cancer and/or the stage of cancer.

### Example 4. Identifying Regions of Genomic DNA Bound to a Transcription Factor Dimer

A biological sample (e.g., cell lysate) comprising genomic DNA bound to a transcription factor dimer is prepared. The genomic DNA is fragmented such that the genomic DNA bound to the transcription factor dimers comprise two free ends. The dimer-bound genomic DNA is extracted from the biological sample with an extraction complex. The extraction complex comprises an extraction moiety (e.g., biotin), oligonucleotide sequences which bind to the free ends of the genomic DNA, cleavage sites near each oligonucleotide sequence, and polynucleotide linkers coupling the oligonucleotides to the biotin. The free ends of the genomic DNA are captured by incubation with biotin-oligonucleotide complex and a DNA ligase such that the oligonucleotide sequences ligate onto the respective free ends of the genomic DNA. The biotinylated DNA-transcription factor dimer complexes are then selectively extracted from the sample using streptavidin-coated microbeads. Following extraction, the oligonucleotide-labeled DNA-transcription factor dimer complexes are released from the extraction complexes by enzyme digestions at the cleavage sites. ChIP is then used to isolate the oligonucleotide-labeled DNA-transcription factor dimer complexes.

Genomic DNA bound to isolated transcription factor dimers is then labeled and purified away from the transcription factors. A solid substrate (e.g., Protein G-coated bead) comprising a first probe (e.g., antibody) with an affinity to a first transcription factor subunit and a second probe (e.g., antibody) with an affinity to a second transcription factor subunit is prepared. The first and second antibodies are further modified to comprise unique first and second tags, respectively. The first and second tags comprise unique polynucleotides (e.g., nucleotide barcodes) with regions for attaching to the first and second ends of the oligonucleotide-labeled DNA, respectively, as well as cleavage sites near each polynucleotide sequence. The isolated oligonucleotide-labeled DNA-transcription factor dimer complexes are incubated with the Protein G beads comprising antibodies specific to two transcription factor subunits of interest. Transcription factor dimers comprising both subunits are captured by the antibodies and proximity ligation is used to ligate the oligonucleotide-labeled DNA ends to the nucleotide barcodes on the tags. The tagged DNA (e.g., barcode-genomic DNA-barcode) is then digested at the cleavage sites to release it from the bead. The tagged DNA is then released from the transcription factors by incubation with Proteinase K and isolated on-column. The isolated and tagged DNA sequence is then amplified (e.g., using PCR with primers specific to the barcode sequences) and sequenced (e.g., using Deep-Seq) in order to identify those regions of the DNA which are bound to transcription factor dimer subunits of interest. Such regions can then be correlated with transcription levels/expression data in order to determine how combinations of transcription factors can affect them.

### Example 5. Nucleic Acid Analysis

Single nucleosomes are prepared, thereafter, a clamp DNA that has two BsaI sites on both ends and a biotin moiety in the middle (extraction moiety) is ligated to the ends of the DNA wrapped around the histone after polishing and TA-sticky ends generation.

The ligated DNA-extraction moiety complex (extraction complex) is purified away from the rest of the mixture by streptavidin beads.

After washing, the nucleosomes on the streptavidin beads are released by BsaI digestion, which generates two different 4-nucliotide sticky ends of the genomic DNA wrapped around the histone. Two antibodies (probes) against two different histone marks are DNA-barcoded, on which they all have a fixed region, a DNA barcoded, and a BsaI site at the distal end of the attached DNA.

The mixture of the two barcoded antibodies are added to the release nucleosome, and are allowed to bind to the modified histone tails The antibody-nucleosome complexes are pulled down with Protein G beads (e.g., sepharose), and after washes the ends of the DNA barcode on two different antibodies are ligated to each sticky end of the genomic DNA wrapped around the histone by adding BsaI and T4 DNA ligase simultaneously forming a ligated DNA product.

After several washes, the ligated DNA products are released from the Protein G beads by either heating, Proteinase K digestion, or a combination of both. Using primers complimentary to the fixed sequences on both ends of the released ligation products, the ligated DNA products are PCR-amplified and deep-sequenced. Using bioinformatics analyses, distributions of the two targeted histone marks will be determined globally at the single nucleosome resolution.

### Example 6. Preparation of a Barcoded Antibody

An antibody comprising an amine is reacted with an oligonucleotide comprising a primer 1 sequence and a 5′-sulfidryl group in the presence of the crosslinker SMCC, crosslinking the sulfidryl of the oligonucleotide to amine of the antibody. The cross-linked antibody is subsequently treated with a barcode oligonucleotide comprising a primer 1 sequence that is complementary to the primer 1 sequence of the crosslinked oligonucleotide, a barcode sequence, and a BsaI restriction sequence. The primer 1 sequence of the cross-linked oligonucleotide hybridizes to the complementary primer 1 sequence of the barcode oligonucleotide to form an annealed product. The 3′ end of the crosslinked oligonucleotide is then extended with a polymerase and nucleotides to form a barcoded antibody comprising a tag comprising a primer sequence, a barcode sequence, and a BsaI restriction sequence.

### Example 7. Determination of a Barcoded Antibody Dilution for Ligation Experiments

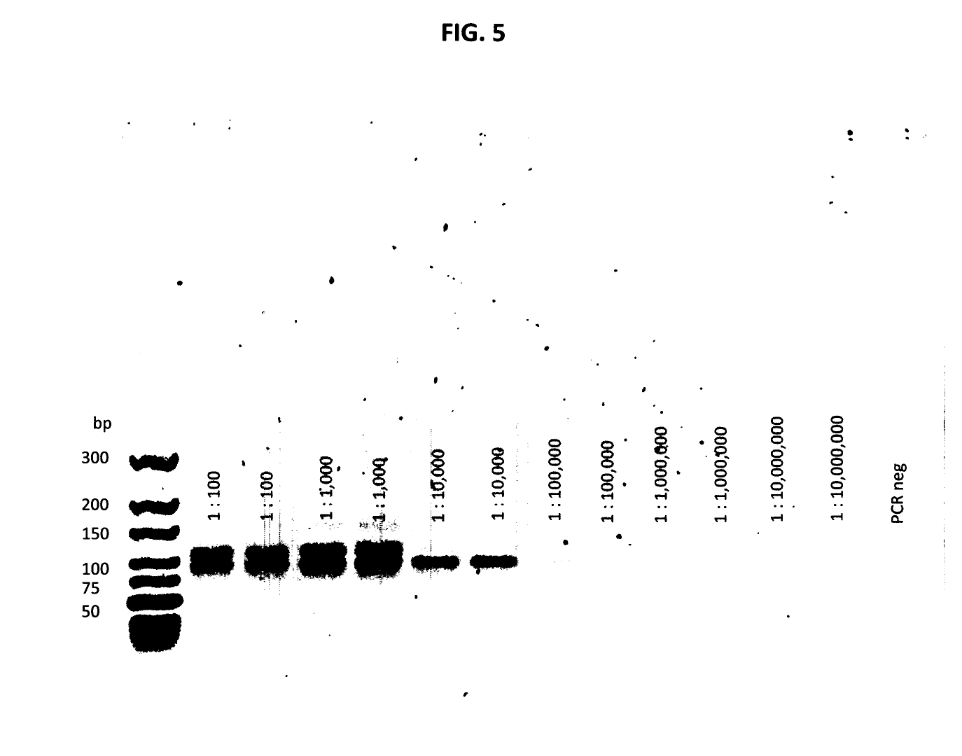

A 1:10 dilution series of set A and set B barcoded antibodies (having compatible ligatable ends following BSA1 digestion) was prepared by diluting a solution of barcoded antibodies by a factor of 1:100, 1:1,000, 1:10,000, 1:1,000,000, and 1:10,000,000. Each dilution was mixed with BsaI and T4 DNA ligase in a total volume of 10 μL, and a BsaI and digestion and T4 DNA ligation reaction was performed. PCR conditions: (37° C.—3 minute; 16° C.—4 minute)×25; 50° C.—5 minute; 4° C. PCR of ligated products was performed by mixing 5 μL from each dilution reaction with 20 μL PCR containing polymerase and nucleotides. 20 cycles of PCR amplification were performed. Products were separated on a 2% agarose gel and imaged with 100V for 1 hr. The results of imaging are shown in FIG. 5. Amplification products at ˜100 bp shown successful ligation despite the absence of dimerized protein targets to bring the barcoded antibodies into proximity for ligation.

### Example 8. Identification of Target Analytes in GM12878 Cells

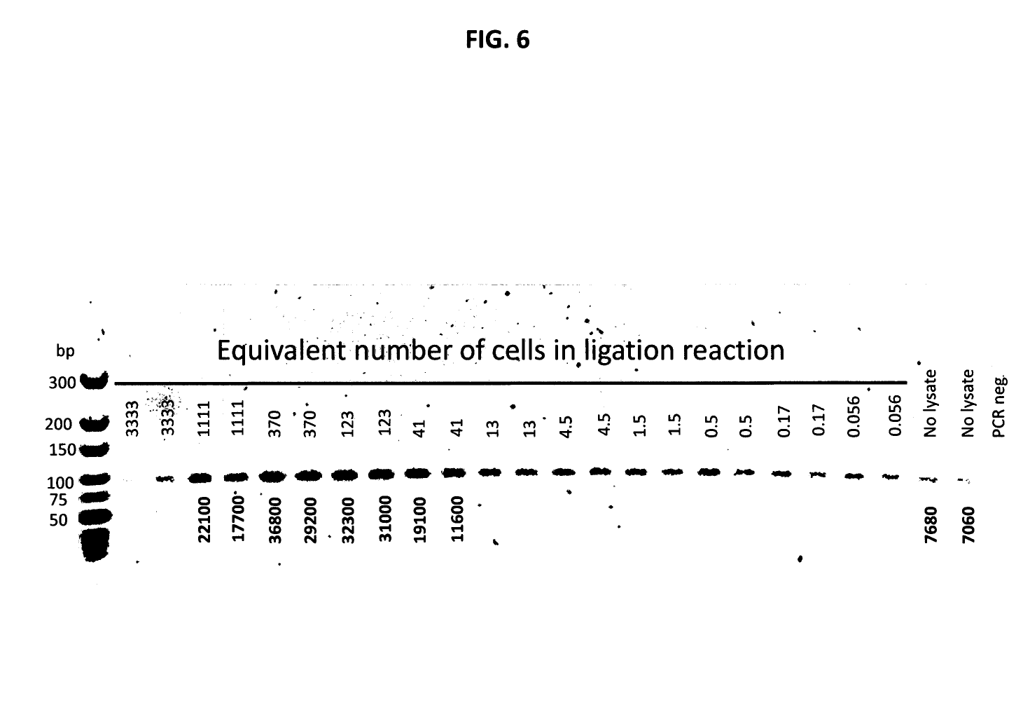

A sample of fixed GM12878 cells was lysed in PBS lysis buffer (1×PBS, 25 mM NaF, 2 mM MgCl₂, 50 μM ZnCl₂, 15% glycerol, 1% triton X-100). The sample was sonicated with a Biorupter Sonicator in 25 cycles of 30 seconds on, and 30 seconds off. A 3-fold dilution series of the lysed sample was prepared (3¹ to 3¹¹) of the cell lysate in a 1× cutsmart (NEB) buffer with set A and set B barcoded antibodies prepared according to the methods disclosed herein and diluted 1:10,000. The resulting samples had a calculated number of cells per sample of 3333, 1111, 370, 123, 41, 13, 4.5, 1.5, 0.5, 0.17, and 0.056. A control sample lacking lysate was also prepared. The lysate-barcode antibody mixtures were incubated at 4° C., and subsequently diluted 1:10, resulting in a total barcoded antibody dilution of 1:100,000. A BsaI/T4 DNA ligation enzyme mixture was added to each of the samples, and the resulting mixtures were incubated for 25 cycles of 3 min. at 37° C. and 4 min. at 16° C., and subsequently incubated for 5 minutes at 50° C., then cooled to 4° C. PCR of ligated products was performed by mixing 5 μL from each dilution reaction with 20 μL PCR containing polymerase and nucleotides. 20 cycles of PCR amplification were performed. Products were separated on a 2% agarose gel at 100V for 1 hr. The results of imaging are shown in FIG. 6. The amplified product visible as a band at ˜100 bp are the result of PCR amplification of a ligation product produced by the binding of set A and set B antibodies to analytes that bring the set A and set B antibodies into proximity with one another (the analytes bound by set A and set B antibodies, respectively, are themselves in proximity, e.g. through dimerization). The numbers 22100, 17700, 36800, 29200, 32300, 31000, 19100, 11600, 7680 and 7060 underneath the 100 bp bands are densitometry results for each band. A ratio relative to the no lysate control was 4.79 for the 370 cell sample, and 4.1 for the 123 cell sample. The results demonstrate that the cell lysate, especially in the sample containing 370 and 123 cells, has more ligated products than the no-lysate control with the same amount of antibodies.

### Example 9. Identification of Target Analytes in GM12878 Cells and Hela Cells

Samples of fixed DH5a cells, GM12878 cells, and Hela cells were lysed in PBS lysis buffer (1×PBS, 25 mM NaF, 2 mM MgCl₂, 50 μM ZnCl₂, 15% glycerol, 1% triton X-100). The samples were sonicated with a Biorupter Sonicator in 25 cycles of 30 seconds on and 30 seconds off. A 3-fold dilution series of each of the lysed samples was prepared (3³ to 3⁶) in a 1× cutsmart (NEB) buffer with set A and set B barcoded antibodies prepared according to the methods disclosed herein and diluted 1:10,000. The resulting dilutions had a calculated number of cells per sample of 37, 12.3, 4.1, and 1.3. A control sample lacking lysate was also prepared. The lysate-barcode antibody mixtures were incubated at 4° C., and subsequently diluted 1:10 or 1:100, resulting two sets of samples with a total barcoded antibody dilution of 1:100,000 and 1:1,000,000, respectively. A BsaI/T4 DNA ligation enzyme mixture was added to each of the samples, and the resulting mixtures were incubated for 25 cycles of 3 min. at 37° C. and 4 min. at 16° C., and subsequently incubated for 5 minutes at 50° C., then cooled to 4° C. PCR of ligated products was performed by mixing 5 μL from each dilution reaction with 20 μL PCR containing polymerase and nucleotides. 20 cycles of PCR amplification were performed. Products were separated on a 2% agarose gel and at 100V for 1 hr. Selected samples were separated on a 10% PAGE gel t 150V for 50 min. and stained with SYBR-gold (1:10,000 dilution for 20 min.)

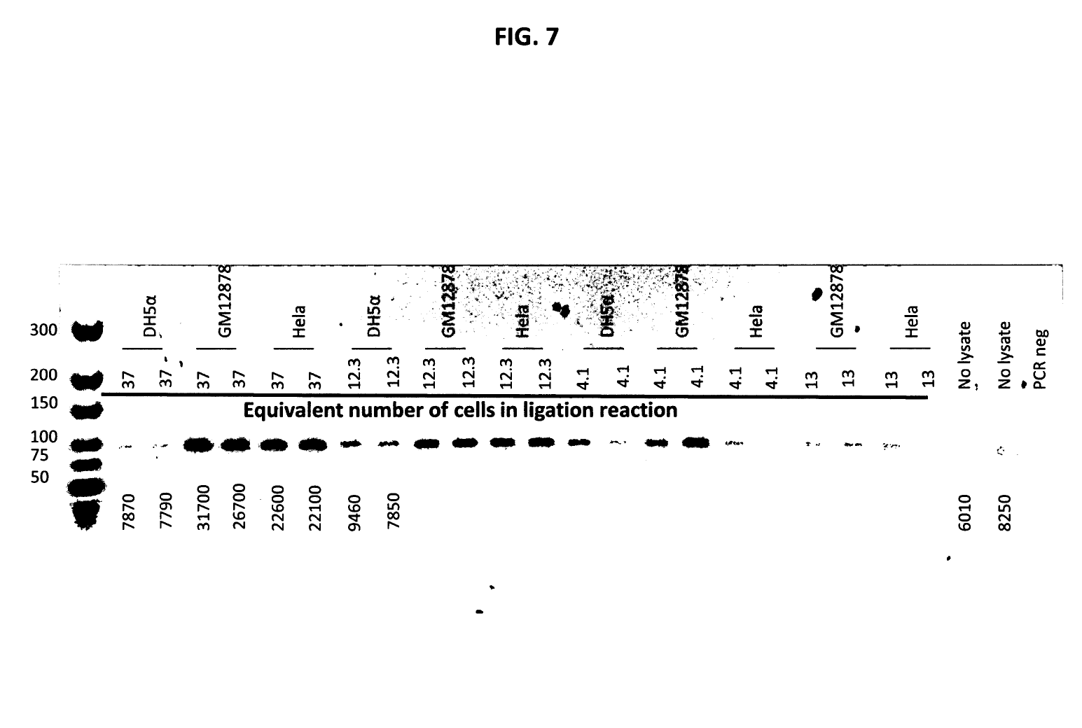

The results of imaging an agarose gel are shown in FIG. 7, for samples with a total antibody dilution of 1:100,000. The amplified products visible as a band at ˜100 bp are the result of PCR amplification of a ligation product produced by the binding of set A and set B antibodies to analytes that bring the set A and set B antibodies into proximity with one another (i.e. the analytes bound by set A and set B antibodies, respectively, are themselves in proximity, e.g. through dimerization). 7870, 7790, 31700, 26700, 22600, 9460, 7850, 6010 and 8250 underneath the 100 bp bands are densitometry results for each band. DH5a cells represented a control, as these bacterial cells lack the dimerizing targets of the set A and set B antibodies. In 37 cell samples, a densitometry ratio relative to the no lysate control was 1.11 for the DH5a control, 4.17 for the GM2878 sample, and 3.19 for the Hela sample.

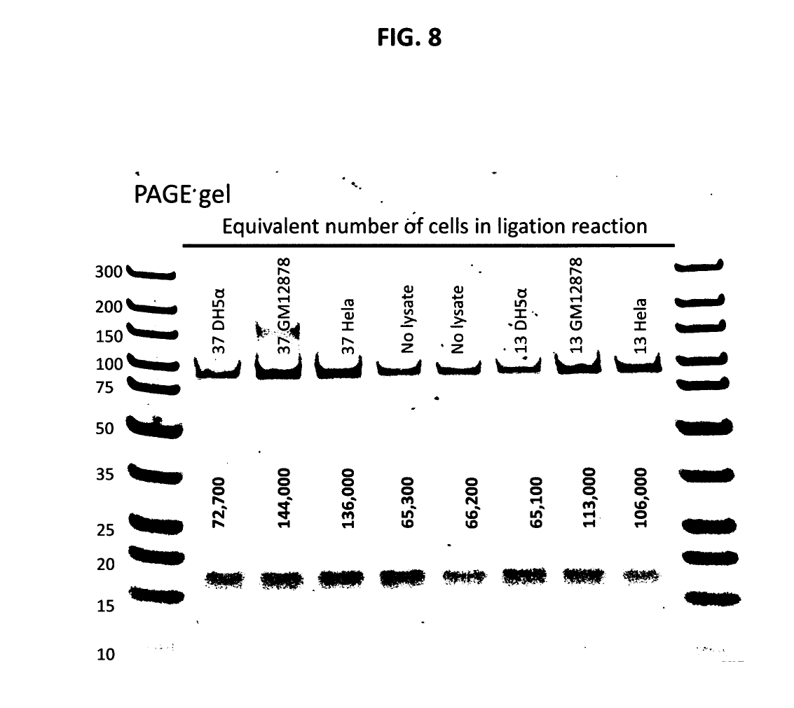

The results of imaging the SDS gel are shown in FIG. 8 for samples with a total antibody dilution of 1:100,000. The densitometry results are noted as 72700, 144000, 136000, 65300, 66200, 65100, 113000, and 106000. In 37 cell samples, a densitometry ratio relative to the no lysate control was 1.11 for the DH5a control, 2.19 for the GM2878 sample, and 2.07 for the Hela sample.

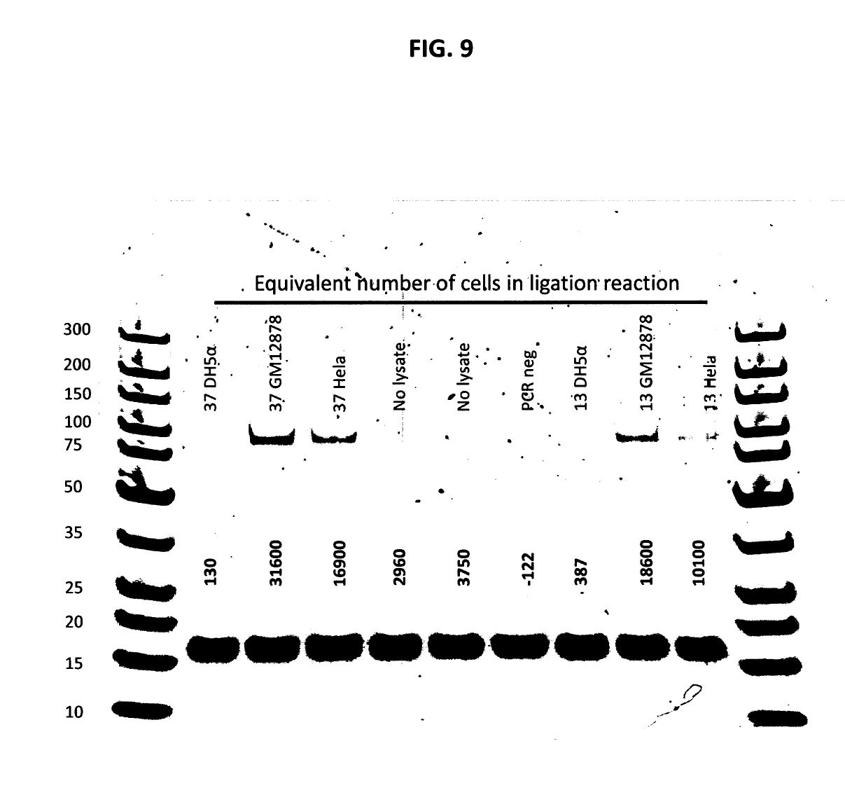

The results of imaging an agarose gel are shown in FIG. 9, for samples with a total antibody dilution of 1:1,000,000. The densitometry results are noted as 130, 31600, 16900, 2960, 3750, −122, 387, 18600 and 10100. In 37 cell-samples, a densitometry ratio relative to the no lysate control was 0.04 for the DH5a control, 9.41 for the GM2878 sample, and 5.04 for the Hela sample. The results demonstrate that the GM2878 and Hela cell lysates have more ligated products than the no-lysate control or DH5α control with the same amount of antibodies.

### Example 10. Identification of Target Analytes in GM12878 Cells

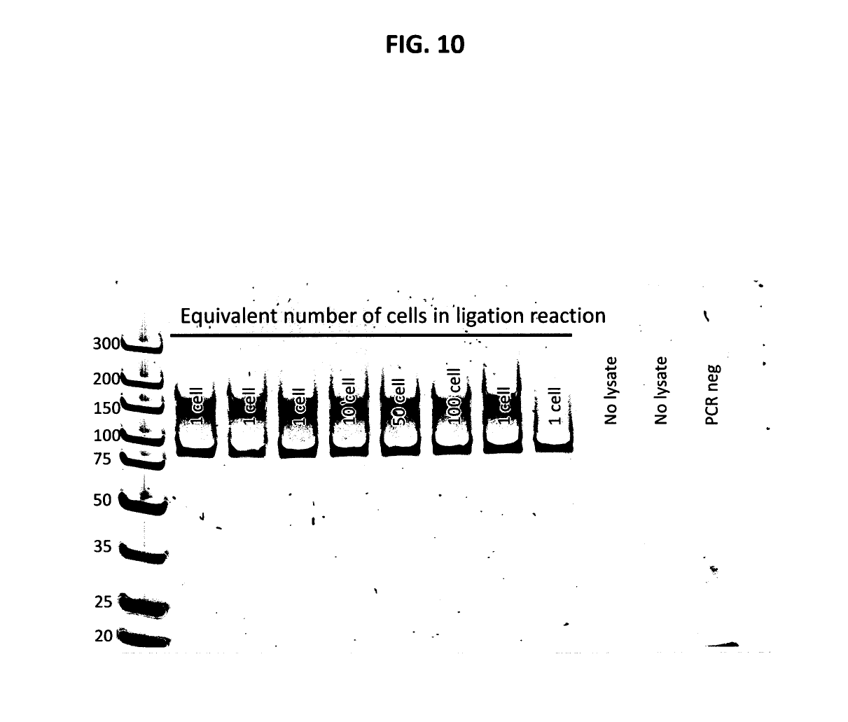

A sample of single cell sorted GM12878 cells was lysed in PBS lysis buffer (1×PBS, 25 mM NaF, 2 mM MgCl2, 50 μM ZnCl2, 15% glycerol, 1% triton X-100). The sample was sonicated with a Biorupter Sonicator in 25 cycles of 30 seconds of sonication, with 30 seconds pause between each sonication (On, Off). A dilution series of each of the lysed samples was prepared in a 1× cutsmart (NEB) buffer with set A and set B barcoded antibodies prepared according to the method disclosed herein and diluted 1:10,000. The resulting dilutions had a calculated number of cells per sample of 1, 10, 50, and 100. A control sample lacking lysate was also prepared. The lysate-barcode antibody mixtures were incubated at 4° C., and subsequently diluted 1:100, resulting in a total barcoded antibody dilution of 1:1,000,000. A BsaI/T4 DNA ligation enzyme mixture was added to each of the samples, and the resulting mixtures were incubated for 25 cycles of 3 min. at 37° C. and 4 min. at 16° C., and subsequently incubated for 5 minutes at 50° C., then cooled to 4° C. PCR of ligated products was performed by mixing 5 from each dilution reaction with 20 μL PCR containing polymerase and nucleotides. 20 cycles of PCR amplification were performed. Products were separated on a 10% PAGE gel t 150V for 50 min. and stained with SYBR-gold (1:10,000 dilution for 20 min.) The results of imaging are shown in FIG. 10. The amplified products visible as a band at ˜100 bp are the result of PCR amplification of a ligation product produced by the binding of set A and set B antibodies to analytes that bring the set A and set B antibodies into proximity with one another (i.e. the analytes bound by set A and set B antibodies, respectively, are themselves in proximity, e.g. through dimerization.) Ligation products are seen for each of the 1, 10, 50, and 100-cell samples, but not for no-lysate and PCR-negative controls.

What is claimed is:

1. A composition comprising:
a. a first probe, wherein the first probe comprises a first tag comprising a polynucleotide comprising a region for attaching to a first end of a nucleic acid; and
b. a second probe, wherein the second probe comprises a second tag comprising a polynucleotide comprising a region for attaching to a second end of the nucleic acid,
wherein the first probe has an affinity to a first binding site on an analyte and the second probe has an affinity to a second binding site on the analyte, wherein the first probe and the second probe are in spatial proximity, and
i. wherein the first probe is associated with a substrate;
ii. wherein the second probe is associated with the substrate;
iii. wherein the first probe is associated with the substrate and wherein the second probe is associated with the substrate;
iv. wherein the first tag is double stranded where associated with the first probe;
v. wherein the second tag is double stranded where associated with the second probe;
vi. wherein the first tag is double stranded where associated with the first probe and wherein the second tag is double stranded where associated with the second probe; or
vii. one of (i), (ii), or (iii) and one of (iv), (v) or (vi).

2. The composition of claim 1, wherein the first probe is associated with a solid substrate.

3. The composition of claim 1 or 2, wherein the second probe is associated with the solid substrate.

4. The composition of any one of claim 1, 2, or 3, wherein the solid substrate is planar.

5. The composition of any one of claims 1-4, wherein the solid substrate is an array.

6. The composition of any one of claim 1, 2, or 3, wherein the solid substrate is spherical.

7. The composition of claim 6, wherein the spherical solid substrate is a bead.

8. The composition of claim 7, wherein the bead is a Sepharose bead.

9. The composition of any one of claims 1-7, wherein at least a portion of the solid substrate is coated.

10. The composition of claim 9, wherein at least a portion of the solid substrate is contacted with at least one of a polymer or a first binding partner which has an affinity for a second binding partner.

11. The composition of claim 10 comprising the polymer, wherein the polymer is selected from the group consisting of polyethylene glycol, polymethacrylate, polymethylmethacrylate, polyethylenimine, polyvinyl alcohol, polyvinyl acetate, polystyrene, polyglutaraldehyde, polyacrylamide, agarose, chitosan, alginate, and a combination thereof.

12. The composition of claim 10 comprising the first binding partner which has an affinity for the second binding partner, wherein the first binding partner is selected from the group consisting of immunoglobulin-binding protein, calmodulin, glutathione, glutathione S-transferase (GST), streptavidin, avidin, maltose-binding protein, a His tag, and a combination thereof.

13. The composition of claim 10 comprising the first binding partner which has an affinity for the second binding partner, wherein the second binding partner is selected from the group consisting of immunoglobulin-binding protein, calmodulin, glutathione, glutathione S-transferase (GST), streptavidin, avidin, maltose-binding protein, a His tag, and a combination thereof.

14. The composition of claim 12 or 13, comprising the immunoglobulin-binding protein wherein the immunoglobulin-binding protein is Protein A or Protein G.

15. The composition of claim 11, wherein each of the first probe and the second probe comprise at least one of a binding partner of the polymer or the second binding partner.

16. The composition of claim 15 comprising the second binding partner, wherein the first binding partner is GST and the first probe and the second probe comprise glutathione.

17. The composition of any one of the above claims, wherein the solid substrate is magnetic.

18. The composition of claim 17, wherein the magnetic solid substrate comprises magnetite, maghemitite, FePt, SrFe, iron, cobalt, nickel, chromium dioxide, ferrites, or a mixture thereof.

19. The composition of any one of claims 1-16, wherein the solid substrate is nonmagnetic.

20. The composition of claim 1, wherein the first probe comprises a first antibody or a fragment thereof, and wherein each first antibody or the fragment thereof comprises at least one of a binding partner of the polymer or the second binding partner.

21. The composition of claim 1 or 20, wherein the second probe comprises a second antibody or a fragment thereof, and wherein each second antibody or the fragment thereof comprises at least one of a binding partner of the polymer or the second binding partner.

22. The composition of claim 20 or 21, wherein the first antibody or the second antibody is a monoclonal antibody, a recombinant antibody, a polyclonal antibody, a chimeric antibody, a humanized antibody, a bispecific antibody, or a fragment thereof.

23. The composition of claim 20 or 21, wherein the first antibody or the second antibody is isolated or purified from a hybridoma.

24. The composition of any one of claims 20-23, wherein the first antibody or the fragment thereof is conjugated with the first tag and the second antibody or the fragment thereof is conjugated with the second tag.

25. The composition of claim 1, wherein the first tag is double stranded.

26. The composition of any one of the above claims, wherein the second tag is double stranded.

27. The composition of any one of claims 1-25, wherein the second tag is single stranded.

28. The composition of any one of the above claims, wherein the first tag comprises a first cleavage site.

29. The composition of any one of the above claims, wherein the second tag comprises a second cleavage site.

30. The composition of claim 28 or 29, wherein the first cleavage site and the second cleavage site are endonuclease recognition sites.

31. The composition of claim 30, wherein the endonuclease sites comprises type II endonuclease recognition sites.

32. The composition of claim 31, wherein the type II endonuclease recognition sites are BsaI recognition sites.

33. The composition of any one of the above claims, wherein the first tag comprises a first barcode.

34. The composition of any one of the above claims, wherein the second tag comprises a second barcode.

35. The composition of claim 33, wherein the first barcode comprises about 1 to 50 nucleotides.

36. The composition of claims 33, 34 or 35, wherein the second barcode comprises about 1 to 50 nucleotides.

37. The composition of claim 33, wherein the first tag comprises a first primer binding site, and the second tag comprises a second primer binding site.

38. The composition of any one of claims 33-37, wherein the first probe is uniquely identifiable by the first barcode.

39. The composition of any one of claims 33-38, wherein the second probe is uniquely identifiable by the second barcode.

40. The composition of any one of claims 1-39, wherein the first polynucleotide and the second polynucleotide are DNA.

41. The composition of any one of claims 1-39, wherein the first polynucleotide and the second polynucleotide are RNA.

42. The composition of any one of claims 1-39, wherein the first polynucleotide and the second polynucleotide are a hybrid of DNA and RNA.

43. The composition of any of the above claims, wherein the analyte comprise a first biological molecule.

44. The composition of claim 43, wherein the first biological molecule is a protein, a carbohydrate, a lipid, or a nucleic acid.

45. The composition of claim 44, wherein the analyte comprises a first protein.

46. The composition of claim 45, wherein the first protein comprises a first modified residue and a second modified residue.

47. The composition of claim 46, wherein the first probe binds to an antigen comprising the first modified residue and the second probe binds to an antigen comprising the second modified residue.

48. The composition of claim 46 or 47, wherein modification on the first modified residue is methylation, phosphorylation, acetylation, ubiquitylation, sumoylation, or a combination thereof.

49. The composition of any one of claims 46-48, wherein modification on the second modified residue is methylation, phosphorylation, acetylation, ubiquitylation, sumoylation, or a combination thereof.

50. The composition of any one of claims 46-49, wherein the first protein is a histone.

51. The composition of claim 50, wherein the histone is modified.

52. The composition of claim 51, wherein the modification is methylation, acetylation, or a combination thereof.

53. The composition of claim 50 or 51, wherein the histone is histone 3.

54. The composition of any one of claims 50-53, wherein the histone is modified at a lysine residue.

55. The composition of any one of the above claims, wherein the analyte further comprises a second protein.

56. The composition of claim 55, wherein the first protein or the second protein comprises a transcription factor.

57. The composition of claim 55 or 56, wherein the first protein and the second protein form a dimer.

58. The composition of any one of claims 55-57, wherein the first protein comprises the first binding site and the second protein comprises the second binding site.

59. The composition of any one of the above claims, wherein the analyte is associated with a nucleic acid.

60. The composition of claim 59, wherein the nucleic acid comprises genomic DNA.

61. The composition of claim 59, wherein the nucleic acid is intracellular or extracellular.

62. The method of claim 59, wherein the nucleic acid is RNA, DNA, or a hybrid thereof.

63. The composition of any one of the above claims, wherein the composition is in the form of an array.

64. A method comprising:
contacting a sample comprising a nucleic acid associated with an analyte with
a. a first probe, wherein the first probe comprises a first tag comprising a polynucleotide comprising a region for attaching to a first end of a nucleic acid; and
b. a second probe, wherein the second probe comprises a second tag comprising a polynucleotide comprising a region for attaching to a second end of the nucleic acid,
wherein the first probe has an affinity to a first binding site on the analyte and the second probe has an affinity to a second binding site on the analyte, wherein the first probe and the second probe are in spatial proximity, and
i. wherein the first probe is associated with a substrate;
ii. wherein the second probe is associated with the substrate;
iii. wherein the first probe is associated with the substrate and wherein the second probe is associated with the substrate;
iv. wherein the first tag is double stranded where associated with the first probe;
v. wherein the second tag is double stranded where associated with the second probe;
vi. wherein the first tag is double stranded where associated with the first probe and wherein the second tag is double stranded where associated with the second probe; or
vii. one of (i), (ii), or (iii) and one of (iv), (v) or (vi).

65. A method comprising:
a. extracting an analyte with a nucleic acid associated with the analyte from a sample by contacting the sample with an extraction complex comprising an extraction moiety and an oligonucleotide, wherein the extraction complex binds to the nucleic acid; and
b. contacting the extracted analyte with:
i. a first probe that has an affinity to a first binding site on the analyte, and
ii. a second probe that has an affinity to a second binding site on the analyte, wherein the first probe comprises a first tag comprising a first polynucleotide comprising a region for attaching to a first end of the nucleic acid, and the second probe comprises a second tag comprising a second polynucleotide comprising a region for attaching to a second end of the nucleic acid, and wherein the first probe and the second probe are in spatial proximity.

66. A method of claim 65, further comprising calculating, with one or more computer processors, a first value of at least one parameter, corresponding to a transcriptional efficiency of at least a portion of the nucleic acid associated with the analyte, and wherein the transcriptional efficiency is correlated to a presence of at least one of the first binding site or the second binding site on the analyte.

67. A method of claim 66, further comprising comparing, with the use of one or more computer processors, the first value of the at least one parameter to a reference value.

68. A method of claim 67, further comprising identifying, with the use of one or more computer processors, a disease in the subject if the first value of the first parameter exceeds the reference value.

69. The method of any one of claims 64-68, wherein the sample is a biological sample.

70. The method of claim 69, wherein the biological sample is selected from the group consisting of amniotic fluid, blood plasma, blood serum, breast milk, cells, cancer cells, tumor cells, cerebrospinal fluid, saliva, semen, synovial fluid, tears, tissue, cancer tissue, tumor tissue, urine, white blood cells, whole blood, and any fraction thereof.

71. A method comprising:
a. associating a substrate to a first probe and a second probe, wherein the first probe comprises a first tag comprising a first polynucleotide and the second probe comprises a second tag comprising a second polynucleotide, wherein the first probe has an affinity to a first binding site on an analyte in a sample, and the second probe has an affinity to a second binding site on the analyte, wherein the first tag comprises a region for attaching to a first end of a nucleic acid associated with the analyte, and the second tag comprises a region for attaching to a second end of the nucleic acid associated with the analyte.

72. The method of any one of claims 64-71, wherein the nucleic acid is an intracellular nucleic acid.

73. The method of any one of claims 64-71, wherein the nucleic acid is an extracellular nucleic acid.

74. The method of any one of claims 64-73, wherein the nucleic acid is DNA.

75. The method of any one of claims 64-73, wherein the nucleic acid is RNA.

76. The method of any one of claims 64-73, wherein the nucleic acid is a hybrid of DNA and RNA.

77. The method of any one of claims 64-76, further comprising modifying the nucleic acid, wherein the modifying comprises generating a single stranded overhang at the first end of the nucleic acid or at the second end of the nucleic acid.

78. The method of claim 64 or 71, further comprising extracting the nucleic acid associated with the analyte from the sample by contacting the sample with an extraction complex comprising an extraction moiety and an oligonucleotide, wherein the extraction complex binds to the nucleic acid.

79. The method of any one of claims 65-68 or 78, wherein the extraction moiety is biotin or a fragment thereof.

80. The method of any one of claims 65-68 or 78-79, wherein the extraction complex comprises a polynucleotide linker.

81. The method of any one of claims 65-68 or 78-80, wherein the oligonucleotide binds to the nucleic acid associated with the analyte.

82. The method of any one of claims 65-68 or 78-81, further comprising dissociating the nucleic acid associated with the analyte from the extraction complex.

83. The method of any one of claims 64-82, wherein at least one of the first probe binds to the first binding site on the analyte or the second probe binds to the second binding site on the analyte.

84. The method of any one of claims 64-83, further comprising attaching the first tag to the first end of the nucleic acid associated with the analyte and the second tag to the second end of the nucleic acid associated with the analyte.

85. The method of any one of claims 64-84, further comprising analyzing the nucleic acid, wherein analyzing the nucleic acid comprises at least one of amplifying the nucleic acid or sequencing the nucleic acid.

86. The method of claim 85, wherein the sequencing comprises multiplex sequencing.

87. The method of claim 85, wherein the amplifying comprises polymerase chain reaction.

88. The method of any one of claim 64 or 71, wherein the substrate is an array.

89. The method of any one of claim 64 or 71, wherein the substrate is a bead.

90. The method of claim 89, wherein the bead is a Sepharose bead.

91. The method of any one of claims 64-70, wherein the method is at least partially performed as a liquid phase assay.
